(19) United States
(12) Patent Application Publication
(10) Pub. No.: US 2026/0191857 A1
(43) Pub. Date: Jul. 9, 2026

(54) COMPOSITIONS AND METHODS FOR TREATING RADIATION INDUCED BRAIN TISSUE DAMAGE

(71) Applicant: Antinous Technology Company Limited, Macau (CN)

(72) Inventors: Ian Ziyar, Fresno, CA (US); Yun Yin, Macau (CN)

(73) Assignee: Antinous Technology Company Limited, Macau (CN)

(21) Appl. No.: 19/550,876

(22) Filed: Feb. 26, 2026

Related U.S. Application Data
(63) Continuation of application No. PCT/US25/35301, filed on Jun. 25, 2025.
(60) Provisional application No. 63/664,580, filed on Jun. 26, 2024.

(51) Int. Cl. A61K 31/505; A61P 25/00

(52) U.S. Cl. CPC A61K 31/505; A61P 25/00

(57) ABSTRACT

The present disclosure in some aspects relates generally to quinoline compounds such as deuterated derivatives thereof, salt thereof, use thereof, and method of preparation thereof. The present disclosure in some aspects relates generally to compounds and methods of their use for treating radiation-induced brain tissue damage, for instance, as side effects of a radiation therapy for treating cancer in a subject.

### CROSS-REFERENCE TO RELATED APPLICATIONS

This application is a continuation of International Patent Application No. PCT/US2025/035301, filed Jun. 25, 2025, which claims priority to U.S. Provisional Application No. 63/664,580, filed Jun. 26, 2024, the disclosures of which applications are considered part of and are herein incorporated by reference for all purposes.

### FIELD

The present disclosure in some aspects relates to anti-angiogenesis compounds, such as deuterated quinoline derivatives, salt or solvate thereof, use thereof, and method of preparation thereof. The present disclosure in some aspects relates to deuterated compounds as angiogenesis inhibitors (e.g., inhibitors of the VEGF signaling pathway) and methods of their use for treating or preventing a disease or condition associated with angiogenesis, such as wet macular degeneration, diabetic retinopathy, or neovascular glaucoma.

### BACKGROUND

Compound 2-2 is a highly potent VEGFR2 protein kinase inhibitor having a structure of Formula (2-2):

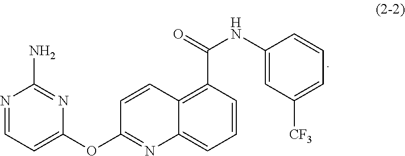 [SMILES: Nc1nccc(Oc2ccc3c(C(=O)Nc4cccc(C(F)(F)F)c4)cccc3n2)n1]

This compound has been shown to exhibit effect against abnormal angiogenesis. However, there remains a need for compounds with improved inhibitory effects on proliferation of angiogenic cells and abnormal angiogenesis.

### SUMMARY

Deuterium (²H or D) is an isope of hydrogen (¹H or H) with a atomic weight twice of that of H. Deuteration process typically refers to substituting some or all of the hydrogen atoms in a drug molecule with deuterium. Due to the similar size and shape of deuterium and hydrogen, the bioactivitiy and/or selectivity of the drug molecule can be sustained after the deuteration. However, C-D bond is more stable than C—H bond, thereby harder to break during the chemical and biological reactions partipated by the drug molecule. As such, deuterated drug molecule can have longer half-life compared to its non-deuterated counterpart.

Deuterating a compound could improve its pharmacokinetic properties, reduce its toxicity and side effects, and thereby facilitate the development of drugs that are potent in treating or preventing a disease or condition. In some embodiments, provided herein are deuterated compounds for treating wet macular degeneration, diabetic retinopathy, neovascular glaucoma, and other eye diseases. In some embodiments, the present disclosure addresses a need for deuterated compound 2-2 and a need for efficient methods to deturate compound 2-2. U.S. Pat. No. 9,540,381 discloses undeuterated compound 2-2, the disclosures of which are incorporated herein by reference in the entirety for all purposes.

In one aspect, provided herein is a compound of structure

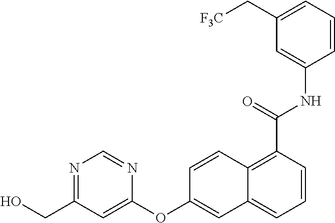 [SMILES: O=C(Nc1cccc(CC(F)(F)F)c1)c1cccc2cc(Oc3cc(CO)ncn3)ccc12]

or of Formula (I), or a pharmaceutically acceptable salt thereof, or a stereoisomer thereof, or a hydrate or a solvate thereof:

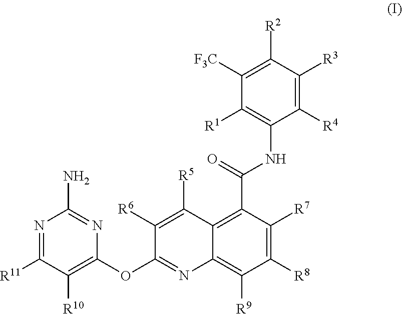

wherein R¹, R², R³, R⁴, R⁵, R⁶, R⁷, R⁸, R⁹, R¹⁰, and R¹¹, are each independently hydrogen or deuterium, provided that at least one or more of R¹, R², R³, R⁴, R⁵, R⁶, R⁷, R⁸, R⁹, R¹⁰, and R¹¹ is deuterium.

In some embodiments, the compound is selected from the group consisting of:

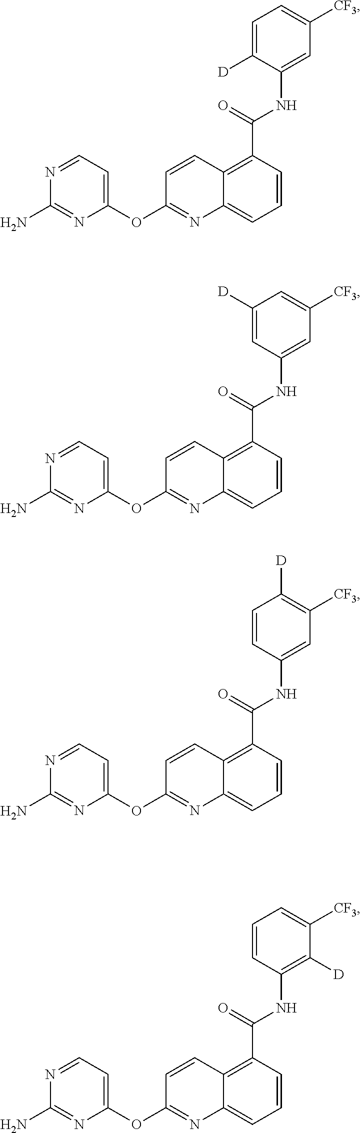 [SMILES: [2H]c1ccc(C(F)(F)F)cc1NC(=O)c1cccc2nc(Oc3ccnc(N)n3)ccc12 ; [2H]c1cc(NC(=O)c2cccc3nc(Oc4ccnc(N)n4)ccc23)cc(C(F)(F)F)c1 ; [2H]c1ccc(NC(=O)c2cccc3nc(Oc4ccnc(N)n4)ccc23)cc1C(F)(F)F ; [2H]c1c(NC(=O)c2cccc3nc(Oc4ccnc(N)n4)ccc23)cccc1C(F)(F)F]

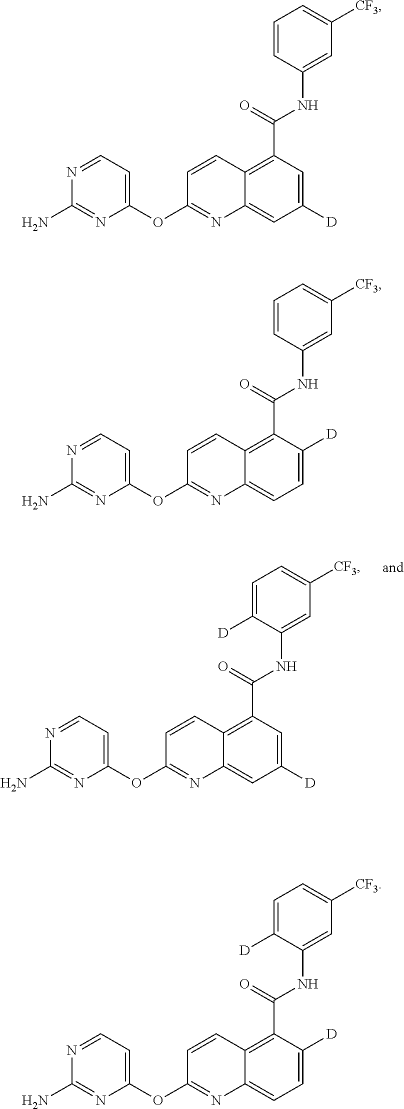 [SMILES: [2H]c1cc(C(=O)Nc2cccc(C(F)(F)F)c2)c2ccc(Oc3ccnc(N)n3)nc2c1 ; [2H]c1ccc2nc(Oc3ccnc(N)n3)ccc2c1C(=O)Nc1cccc(C(F)(F)F)c1 ; [2H]c1cc(C(=O)Nc2cc(C(F)(F)F)ccc2[2H])c2ccc(Oc3ccnc(N)n3)nc2c1 ; [2H]c1ccc(C(F)(F)F)cc1NC(=O)c1c([2H])ccc2nc(Oc3ccnc(N)n3)ccc12]

In one aspect, provided herein is a compound of Formula (II), or a pharmaceutically acceptable salt thereof, or a stereoisomer thereof, or a hydrate or a solvate thereof:

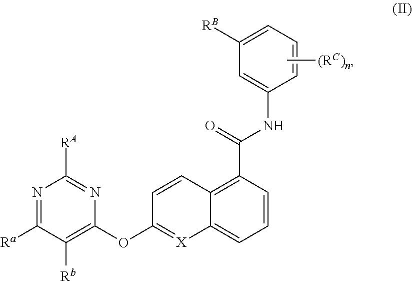

or a pharmaceutically acceptable salt thereof, wherein:
X is N or CH;
R^{A} is H, —N(R^{x})₂, —OR^{x}, —SR, —S(O)R^{x}, —S(O)₂R^{x}, or S(O)₂N(R^{x})₂ or C₁-C₆ alkyl optionally substituted with one or more independently selected —N(R^{x})₂ or —OR^{x};
R^{B} is C₁-C₆ alkyl optionally substituted with one or more halo;
R^{C} is —CR^{c}R^{d}R^{e}, wherein R^{c}, R^{d}, and R^{e} are each independently H, halogen, or C₁-C₆ alkyl optionally substituted with one or more independently selected halogen;
n is an integer from 0 to 4;
R^{a} is H, —N(R^{x})₂, —OR^{x}, —SR^{x}, —S(O)R^{x}, —S(O)₂R^{x}, S(O)₂N(R^{x})₂ or C₁-C₆ alkyl optionally substituted with one or more independently selected —N(R^{x})₂ or —OR^{x};
R^{b} is H or C₁-C₆ alkyl;
or, R^{a} and R^{b} together with the carbon atom connecting them form a phenyl, C₅-C₆ cycloalkyl, 5- to 6-membered heteroaryl, or 5- to 6-membered heterocyclyl, each of which is optionally substituted by one or more independently selected halo or C₁-C₆ alkyl;
wherein R^{x}, at each occurrence, is independently H or C₁-C₆ alkyl; and
wherein one or more of the hydrogen(s) in Formula (II) is optionally replaced by deuterium.

In some embodiments, the compound is selected from the group consisting of:

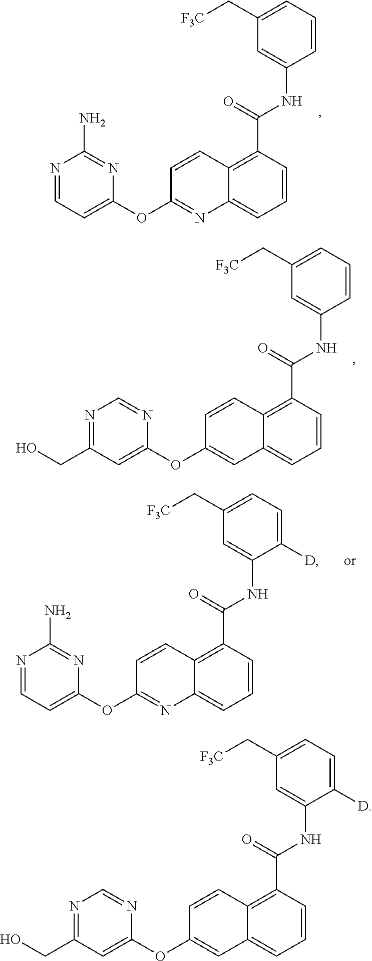

In some embodiments, the compound is

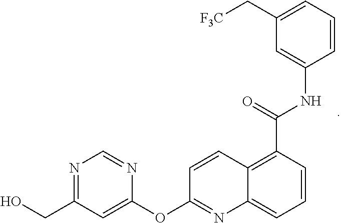 [SMILES: O=C(Nc1cccc(CC(F)(F)F)c1)c1cccc2nc(Oc3cc(CO)ncn3)ccc12]

In some embodiments, the compound is

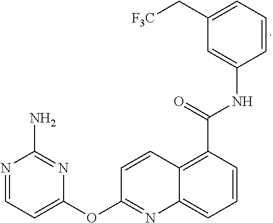 [SMILES: Nc1nccc(Oc2ccc3c(C(=O)Nc4cccc(CC(F)(F)F)c4)cccc3n2)n1]

In some embodiments, the pharmaceutically acceptable salt is selected from the group consisting of phosphate salt, D-camphorsulfonate salt, chloride salt, bromide salt, fluoride salt, sulfate salt, nitrate salt, formate salt, acetate salt, propionate salt, oxalate salt, malonate salt, succinate salt, fumarate salt, maleate salt, lactate salt, malate salt, tartrate salt, citrate salt, picrate salt, methanesulfonate salt, benzene mesylate salt, benzenesulfonate salt, aspartate salt, and glutamate salt.

In some embodiments, provided herein is a pharmaceutical composition comprises the compound of Formula (I) or Formula (II), or a pharmaceutically acceptable salt thereof, or a stereoisomer thereof, or a hydrate or a solvate thereof. In some embodiments, the pharmaceutical composition is an ophthalmic formulation. In some embodiments, the pharmaceutical composition is in the form of eye drop, eye ointment or ophthalmic injectable composition.

In some embodiments, provided herein is use of a compound provided herein, or a pharmaceutically acceptable salt thereof, or a stereoisomer thereof, or a hydrate or a solvate thereof, or the pharmaceutically composition provided herein, in preparation of a medicament for inhibiting abnormal angiogenic cell proliferation or angiogenesis or treating a disease or condition associated with abnormal angiogenic cell proliferation or angiogenesis. In some embodiments, the medicament for inhibiting abnormal angiogenic cell proliferation or angiogenesis is a VEGFR2 inhibitor. In some embodiments, the medicament for inhibiting abnormal angiogenic cell proliferation or angiogenesis is a medicament against ocular angiogenesis and the disease or condition associated with abnormal angiogenic cell proliferation or angiogenesis is a disease or condition associated with ocular angiogenesis. In some embodiments, the medicament for inhibiting abnormal angiogenic cell proliferation or angiogenesis is a choroidal angiogenesis inhibitor and the disease or condition associated with abnormal angiogenic cell proliferation or angiogenesis is a disease or condition associated with choroidal angiogenesis. In some embodiments, the medicament for inhibiting abnormal angiogenic cell proliferation or angiogenesis is a medicament for treating wet macular degeneration, diabetic retinopathy, or neovascular glaucoma, and wherein the disease is wet macular degeneration, diabetic retinopathy, or neovascular glaucoma.

In some aspects, provided herein is a method of improving the efficacy of compound 2-2 in inhibiting VEGFR2 protein kinase

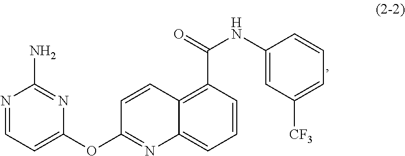 [SMILES: Nc1nccc(Oc2ccc3c(C(=O)Nc4cccc(C(F)(F)F)c4)cccc3n2)n1]

comprising substituting at least one hydrogen in compound 2-2 with deuterium. In some embodiments, the hydrogen is a hydrogen connected to an aromatic ring or aromatic bicyclic ring in compound 2-2. In some embodiments, the hydrogen is at the para position of the —CF₃ group.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 is a ¹H NMR spectrum of Compound 1.

FIG. 2 shows the level of autophosphorylation of VEGF receptor2/KDR when treated with Compound 1.

FIG. 3 shows the comparison of VEGFR inhibition efficacy between Compound 1 and undeuterated counterpart of Compound 1.

FIG. 4A shows the therapeutic efficacy of Compound HY-3-2-H (also referred to as compound KR2) in non-small cell lung cancer (NSCLC) patient-derived xenograft (PDX) model after treatment with Compound HY-3-2-H (60 mg/kg), measured by changes in tumor volume.

FIG. 4B shows the therapeutic efficacy of HY-3-2-H in NSCLC PDX model after treatment with HY-3-2-H (60 mg/kg), measured by changes in tumor weight.

FIG. 4C shows the effect of compound HY-3-2-H in NSCLC PDX model after treatment with HY-3-2-H (60 mg/kg), measured by changes in body weight of NSCLC PDX bearing mice.

FIG. 5A shows the therapeutic efficacy of HY-3-2-H in hepatocellular carcinoma (HCC) PDX model after treatment with HY-3-2-H (60 mg/kg), measured by changes in tumor volume.

FIG. 5B shows the therapeutic efficacy of HY-3-2-H in HCC PDX model after treatment with HY-3-2-H (60 mg/kg), measured by changes in tumor weight.

FIG. 5C shows the effect of HY-3-2-H in HCC PDX model after treatment with HY-3-2-H (60 mg/kg), measured by changes in body weight of HCC PDX bearing mice.

FIG. 6A shows an experimental design of an anti-VEGF comparative therapy with the compound ANT-VI-4-1 (also referred to as HY-3-2-H) or anti-VEGF antibody Bevacizumab.

FIG. 6B shows the therapeutic efficacy of the compound ANT-VI-4-1 or anti-VEGF antibody Bevacizumab in the anti-VEGF comparative therapy, measured by infiltrating CD8⁺ T cells.

FIG. 6C shows the therapeutic efficacy of the compound ANT-VI-4-1 or anti-VEGF antibody Bevacizumab in the anti-VEGF comparative therapy, measured by GFAP (Glial Fibrillary Acidic Protein) and IBA1 (Ionized Calcium-Binding Adapter Molecule 1) immunoreactivities for microglia and macrophages activities after radiation.

FIG. 7A shows the therapeutic efficacy of the compound ANT-VI-4-1 or anti-VEGF antibody Bevacizumab in the anti-VEGF comparative therapy, measured by NeuN (Neuronal Nuclei) immunoreactivity for surviving neurons and TUNEL (Terminal deoxynucleotidyl transferase dUTP Nick End Labeling) immunoreactivity for DNA fragmentation after radiation.

FIG. 7B shows the therapeutic efficacy of the compound ANT-VI-4-1 or anti-VEGF antibody Bevacizumab in the anti-VEGF comparative therapy, measured by fibrinogen immunoreactivity for hemostasis activity after radiation.

FIG. 8 shows the therapeutic efficacy of the compound ANT-VI-4-1 or anti-VEGF antibody Bevacizumab in the anti-VEGF comparative therapy, measured by GLUT1 (Glucose Transporter Type 1) immunoreactivity for blood flow recovery and proper vascular network formation after radiation.

FIG. 9 shows that phosphorylation of VEGFR2/KDR was significantly inhibited by KDR2-2 (also referred to as Compound 2-2), HY-3-2-H, and Compound 1.

FIG. 10 shows that HY-3-2-H, KDR2-2 (Compound 2-2), and Compound 1 inhibited the migration of HUVEC cells.

FIGS. 11A-11B show that HY-3-2-H, KDR2-2 (Compound 2-2), and Compound 1 inhibited tube formation capability, reducing both the total tube length (FIG. 11A) and number of nodes (FIG. 11B) in HUVEC cells.

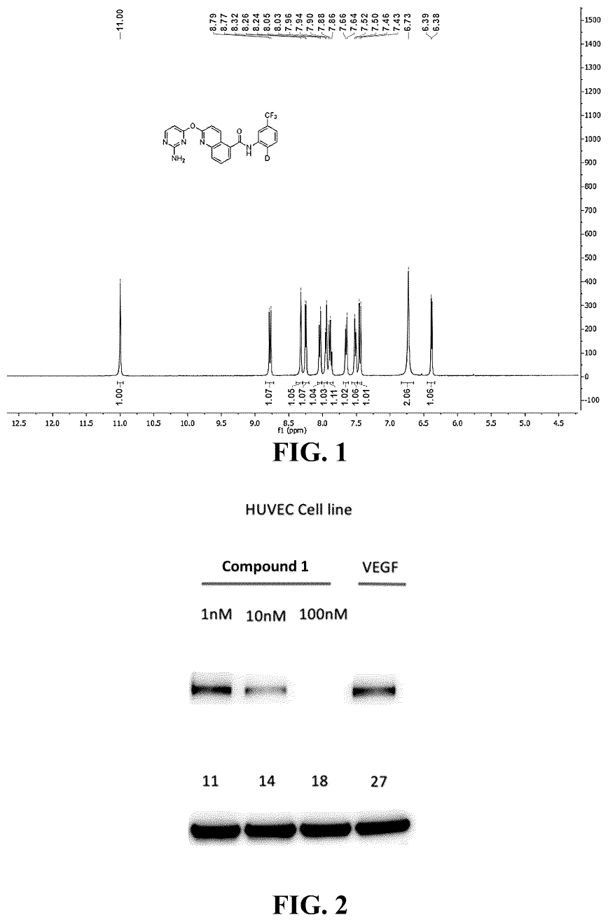

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### I. Definition

As used in the present specification, the following words and phrases are generally intended to have the meanings as set forth below, except to the extent that the context in which they are used indicates otherwise.

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, where applicable, the terms “about” and “approximately,” when used in this context, contemplate a dose, amount, or weight percent within 15 of the specified dose, amount, or weight percent.

It is understood that embodiments described herein as “comprising” include “consisting of” and “consisting essentially of” embodiments.

Unless indicated otherwise, “an individual” or “a subject” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods provided herein contemplate any one or more of these aspects of treatment.

In certain instances, the terms “prevention”, “prophylaxis” and “preclusion” are used synonymously and refer to the avoidance or reduction of the risk of contracting, experiencing, suffering from or having a disease, a condition, a disorder (e.g., abnormal angiogenic cell proliferation or angiogenesis), a symptom or a health problem, or a development or advancement of such states and/or the symptoms of such states. In certain instances, prevention means delaying the development of a disease or any symptom thereof. For example, prevention of a proliferative disorder means to delay, defer, hinder, slow, retard, stabilize, and/or postpone development of the disorder or any symptom thereof. This delay can be of varying lengths of time, depending on the history of the disease and/or individual being treated. As is evident to one skilled in the art, a sufficient or significant prevention or delay can, in effect, result in that the individual does not develop the disease. A method that prevents development of a abnormal angiogenic cell proliferation or angiogenesis is a method that reduces probability of disease development in a given time frame and/or reduces the extent of the disorder in a given time frame, when compared to not using the method. Such comparisons are typically based on clinical studies, using a statistically significant number of subjects. Development may also refer to disease progression that may be initially undetectable and includes occurrence, recurrence, and onset. The treatment or prevention of a disease, a condition, a disorder, an injury or a health problem may be partial or complete.

The term “effective amount” as used herein, refers to a sufficient amount of at least one agent being administered to achieve a desired result, e.g., to inhibit abnormal angiogenic cell proliferation or angiogenesis or to relieve to some extent one or more symptoms of a disease or condition being treated. In certain instances, the method is in vitro, and the desired result may comprise certain desired alteration of cells or biological processes. In certain instances, the method is in vivo, and the result may comprise a reduction and/or alleviation of the signs, symptoms, or causes of a disease, such as abnormal angiogenic cell proliferation or angiogenesis. In certain instances, the result is a death of or decrease in the growth of at least one abnormally proliferating cell. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

In certain instances, an “effective amount” is considered in the context of therapeutical uses and may be optionally referred to as “therapeutically effective amount”. A “therapeutically effective amount” refers to an amount of the compound or the composition comprising a compound or salt thereof as set forth herein sufficient to produce a desired therapeutic outcome and/or required to provide a clinically significant decrease in a disease. An appropriate “effective” or “therapeutically effective” amount in any individual case is determined using any suitable technique, such as a dose escalation study. In various embodiments, an effective amount or a therapeutically effective amount of the compound may (i) reduce the number of abnormal angiogenic cell proliferation or angiogenesis; (ii) reduce symptoms associated with abnormal angiogenic cell proliferation or angiogenesis such as wet macular degeneration; (iii) inhibit, retard, slow to some extent, and preferably stop abnormal angiogenic cell proliferation or angiogenesis; (iv) inhibit (e.g., slow to some extent and preferably stop) abnormal angiogenic cell proliferation or angiogenesis; and/or (v) prevent or delay occurrence and/or recurrence of abnormal angiogenic cell proliferation or angiogenesis. In various embodiments, the amount is sufficient to ameliorate, palliate, lessen, and/or delay one or more of symptoms of wet macular degeneration, diabetic retinopathy, or neovascular glaucoma.

### II. Compound

In some aspects, provided herein is a compound of Formula (I), or a pharmaceutically acceptable salt thereof, or a stereoisomer thereof, or a hydrate or a solvate thereof:

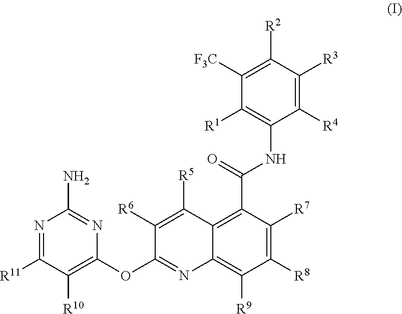

wherein R¹, R², R³, R⁴, R⁵, R⁶, R⁷, R⁸, R⁹, R¹⁰, and R¹¹, are each independently hydrogen or deuterium, provided that at least one of R¹, R², R³, R⁴, R⁵, R⁶, R⁷, R⁸, R⁹, R¹⁰, and R¹¹ is deuterium.

In some embodiments of Formula (I), R¹, R², R³, and R⁴ are each independently hydrogen or deuterium, R⁶, R⁷, R⁸, R⁹, R¹⁰, and R¹¹, are each hydrogen, provided that at least one of R¹, R², R³, and R⁴ is deuterium. In some embodiments, one, two, three, or four of R¹, R², R³, and R⁴ are deuterium. In some embodiments, one or two of R² and R⁴ is deuterim and R³ is H.

In some embodiments of Formula (I), R⁵, R⁶, R⁷, R⁸, and R⁹ are each independently hydrogen or deuterium, R¹, R², R³, R⁴, R¹⁰, and R¹¹, are each hydrogen, provided that at least one of R⁵, R⁶, R⁷, R⁸, and R⁹ is deuterium. In some embodiments, one, two, three, four, or five of R⁵, R⁶, R⁷, R⁸, and R⁹ are deuterium. In some embodiments, one or two of R⁵ and R⁶ is deuterim and R⁷, R⁸, and R⁹ are each H. In some embodiments, one or two or three of R⁷, R⁸, and R⁹ is deuterium and R⁵ and R⁶ are both H.

In some embodiments of Formula (I), R¹⁰ and R¹¹ are each independently hydrogen or deuterium, R¹, R², R³, R⁴, R⁵, R⁶, R⁷, R⁸, and R⁹, are each hydrogen, provided that at least one of R¹⁰ and R¹¹ is deuterium. In some embodiments, one or two of R¹⁰ and R¹¹ are deuterium.

In some embodiments, R¹ is deuterium and R², R³, R⁴, R⁵, R⁶, R⁷, R⁸, R⁹, R¹⁰, and R¹¹ are hydrogen. In some embodiments, R² is deuterium and R¹, R³, R⁴, R⁵, R⁶, R⁷, R⁸, R⁹, R¹⁰, and R¹¹ are hydrogen. In some embodiments, R³ is deuterium and R¹, R², R⁴, R⁵, R⁶, R⁷, R⁸, R⁹, R¹⁰, and R¹¹ are hydrogen. In some embodiments, R⁴ is deuterium and R¹, R², R³, R⁵, R⁶, R⁷, R⁸, R⁹, R¹⁰, and R¹¹ are hydrogen. In some embodiments, R⁵ is deuterium and R¹, R², R³, R⁴, R⁶, R⁷, R⁸, R⁹, R¹⁰, and R¹¹ are hydrogen. In some embodiments, R⁶ is deuterium and R¹, R², R³, R⁴, R⁵, R⁷, R⁸, R⁹, R¹⁰, and R¹¹ are hydrogen. In some embodiments, R⁷ is deuterium and R¹, R², R³, R⁴, R⁵, R⁶, R⁸, R⁹, R¹⁰, and R¹¹ are hydrogen. In some embodiments, R⁸ is deuterium and R¹, R², R³, R⁴, R⁵, R⁶, R⁷, R⁹, R¹⁰, and R¹¹ are hydrogen. In some embodiments, R⁹ is deuterium and R¹, R², R³, R⁴, R⁵, R⁶, R⁷, R⁸, R¹⁰, and R¹¹ are hydrogen. In some embodiments, R¹⁰ is deuterium and R¹, R², R³, R⁴, R⁵, R⁶, R⁷, R⁸, R⁹, and R¹¹ are hydrogen. In some embodiments, R¹¹ is deuterium and R¹, R², R³, R⁴, R⁵, R⁶, R⁷, R⁸, R⁹, and R¹⁰ are hydrogen.

In some embodiments of Formula (I), R¹ is deuterium, and at least one (e.g., any of 1, 2, 3, 4, 5, 6, 7, 8, 9, or 10) of R², R³, R⁴, R⁵, R⁶, R⁷, R⁸, R⁹, R¹⁰, and R¹¹ is deuterium. In some embodiments, R² is deuterium, and at least one (e.g., any of 1, 2, 3, 4, 5, 6, 7, 8, 9, or 10) of R¹, R³, R⁴, R⁵, R⁶, R⁷, R⁸, R⁹, R¹⁰, and R¹¹ is deuterium. In some embodiments, R³ is deuterium, and at least one (e.g., any of 1, 2, 3, 4, 5, 6, 7, 8, 9, or 10) of R¹, R², R⁴, R⁵, R⁶, R⁷, R⁸, R⁹, R¹⁰, and R¹¹ is deuterium. In some embodiments, R⁴ is deuterium, and at least one (e.g., any of 1, 2, 3, 4, 5, 6, 7, 8, 9, or 10) of R¹, R², R³, R⁵, R⁶, R⁷, R⁸, R⁹, R¹⁰, and R¹¹ is deuterium. In some embodiments, R⁵ is deuterium, and at least one (e.g., any of 1, 2, 3, 4, 5, 6, 7, 8, 9, or 10) of R¹, R², R³, R⁴, R⁶, R⁷, R⁸, R⁹, R¹⁰, and R¹¹ is deuterium. In some embodiments, R⁶ is deuterium, and at least one (e.g., any of 1, 2, 3, 4, 5, 6, 7, 8, 9, or 10) of R¹, R², R³, R⁴, R⁵, R⁷, R⁸, R⁹, R¹⁰, and R¹¹ is deuterium. In some embodiments, R⁷ is deuterium, and at least one (e.g., any of 1, 2, 3, 4, 5, 6, 7, 8, 9, or 10) of R¹, R², R³, R⁴, R⁵, R⁶, R⁸, R⁹, R¹⁰, and R¹¹ is deuterium. In some embodiments, R⁸ is deuterium, and at least one (e.g., any of 1, 2, 3, 4, 5, 6, 7, 8, 9, or 10) of R¹, R², R³, R⁴, R⁵, R⁶, R⁷, R⁹, R¹⁰, and R¹¹ is deuterium. In some embodiments, R⁹ is deuterium, and at least one (e.g., any of 1, 2, 3, 4, 5, 6, 7, 8, 9, or 10) of R¹, R², R³, R⁴, R⁵, R⁶, R⁷, R⁸, R¹⁰, and R¹¹ is deuterium. In some embodiments, R¹⁰ is deuterium, and at least one (e.g., any of 1, 2, 3, 4, 5, 6, 7, 8, 9, or 10) of R¹, R², R³, R⁴, R⁵, R⁶, R⁷, R⁸, R⁹, and R¹¹ is deuterium. In some embodiments, R¹¹ is deuterium, and at least one (e.g., any of 1, 2, 3, 4, 5, 6, 7, 8, 9, or 10) of R¹, R², R³, R⁴, R⁵, R⁶, R⁷, R⁸, R⁹, and R¹⁰ is deuterium.

In some embodiments, the compound of formula (I) is selected from the group consisting

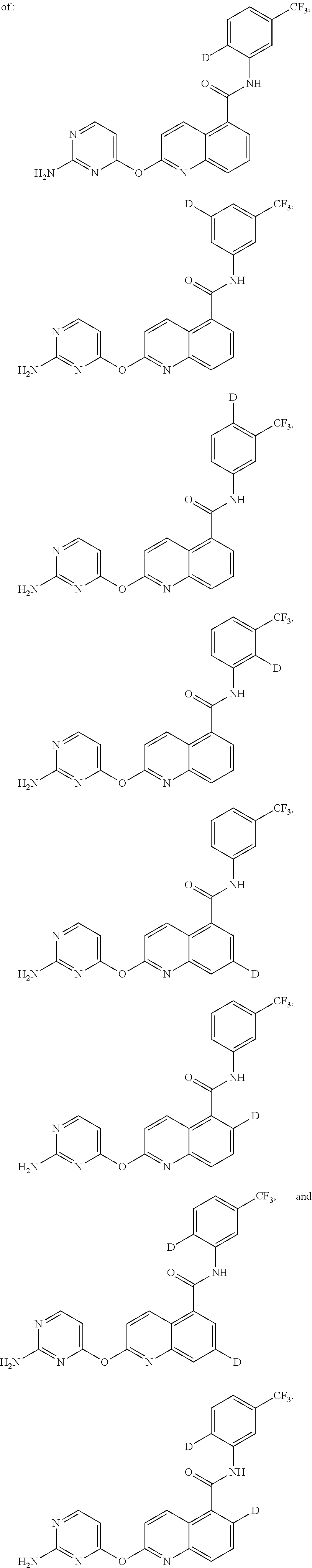

In some embodiments, provided herein is a compound selected from Table 1 and Table 2.

TABLE 1

| Compound | |
|---|---|
| No. | Structure |
| 1 | 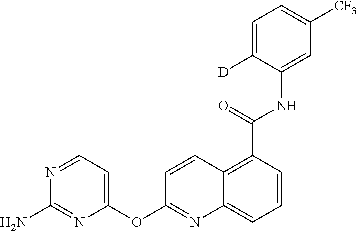 [SMILES: [2H]c1ccc(C(F)(F)F)cc1NC(=O)c1cccc2nc(Oc3ccnc(N)n3)ccc12] |
| 2 | 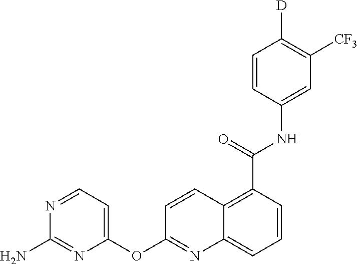 [SMILES: [2H]c1ccc(NC(=O)c2cccc3nc(Oc4ccnc(N)n4)ccc23)cc1C(F)(F)F] |
| 3 | 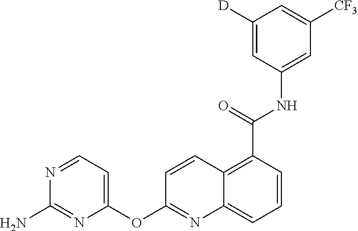 [SMILES: [2H]c1cc(NC(=O)c2cccc3nc(Oc4ccnc(N)n4)ccc23)cc(C(F)(F)F)c1] |
| 4 | 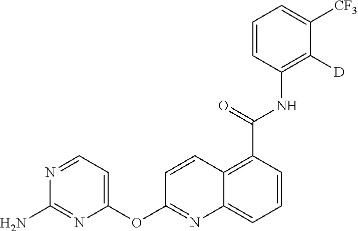 [SMILES: [2H]c1c(NC(=O)c2cccc3nc(Oc4ccnc(N)n4)ccc23)cccc1C(F)(F)F] |
| 5 | 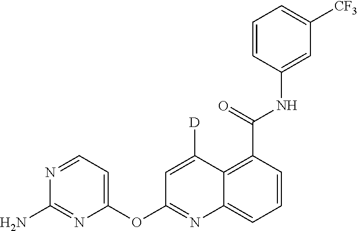 [SMILES: [2H]c1cc(Oc2ccnc(N)n2)nc2cccc(C(=O)Nc3cccc(C(F)(F)F)c3)c12] |
| 6 | 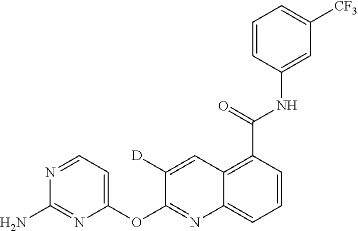 [SMILES: [2H]c1cc2c(C(=O)Nc3cccc(C(F)(F)F)c3)cccc2nc1Oc1ccnc(N)n1] |
| 7 | 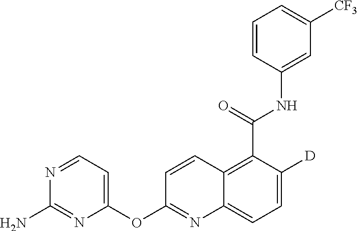 [SMILES: [2H]c1ccc2nc(Oc3ccnc(N)n3)ccc2c1C(=O)Nc1cccc(C(F)(F)F)c1] |
| 8 | 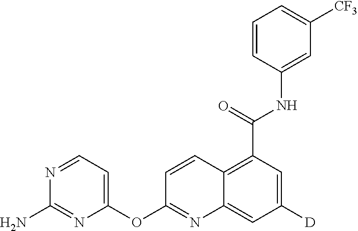 [SMILES: [2H]c1cc(C(=O)Nc2cccc(C(F)(F)F)c2)c2ccc(Oc3ccnc(N)n3)nc2c1] |
| 9 | 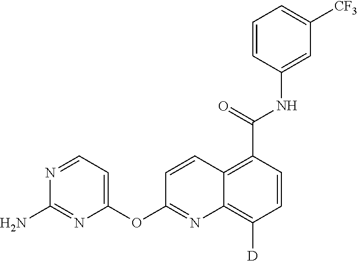 [SMILES: [2H]c1ccc(C(=O)Nc2cccc(C(F)(F)F)c2)c2ccc(Oc3ccnc(N)n3)nc12] |
| 10 | 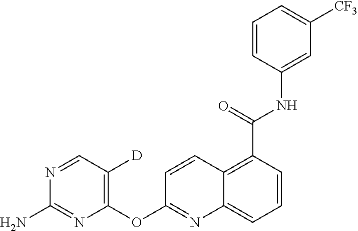 [SMILES: [2H]c1cnc(N)nc1Oc1ccc2c(C(=O)Nc3cccc(C(F)(F)F)c3)cccc2n1] |
| 11 | 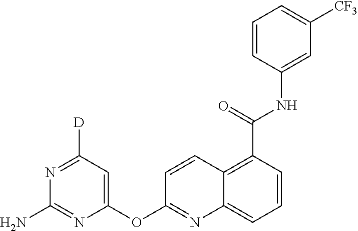 [SMILES: [2H]c1cc(Oc2ccc3c(C(=O)Nc4cccc(C(F)(F)F)c4)cccc3n2)nc(N)n1] |

TABLE 2

| Compound | |
|---|---|
| No. | Structure |
| 2-1 | 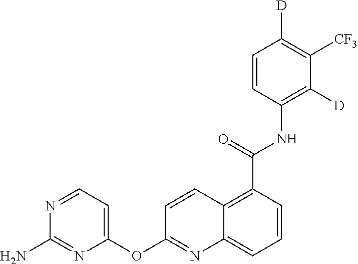 [SMILES: [2H]c1ccc(NC(=O)c2cccc3nc(Oc4ccnc(N)n4)ccc23)c([2H])c1C(F)(F)F] |
| 2-2 | 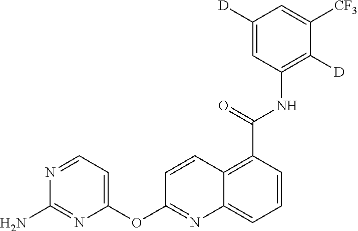 [SMILES: [2H]c1cc(NC(=O)c2cccc3nc(Oc4ccnc(N)n4)ccc23)c([2H])c(C(F)(F)F)c1] |
| 2-3 | 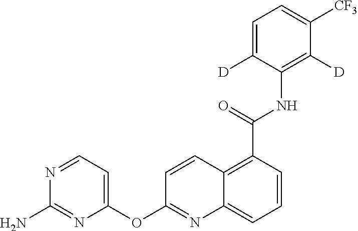 [SMILES: [2H]c1ccc(C(F)(F)F)c([2H])c1NC(=O)c1cccc2nc(Oc3ccnc(N)n3)ccc12] |
| 2-4 | 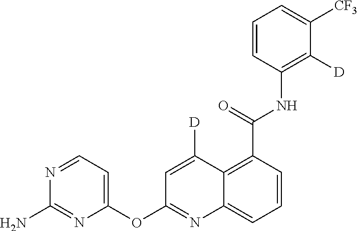 [SMILES: [2H]c1c(NC(=O)c2cccc3nc(Oc4ccnc(N)n4)cc([2H])c23)cccc1C(F)(F)F] |
| 2-5 | 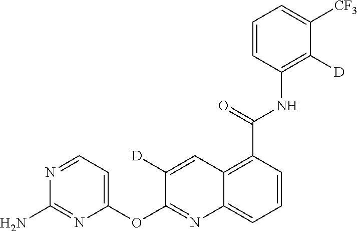 [SMILES: [2H]c1cc2c(C(=O)Nc3cccc(C(F)(F)F)c3[2H])cccc2nc1Oc1ccnc(N)n1] |
| 2-6 | 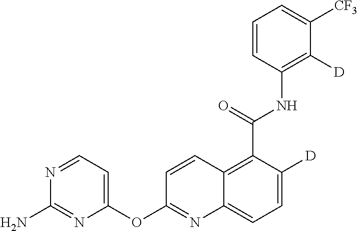 [SMILES: [2H]c1ccc2nc(Oc3ccnc(N)n3)ccc2c1C(=O)Nc1cccc(C(F)(F)F)c1[2H]] |
| 2-7 | 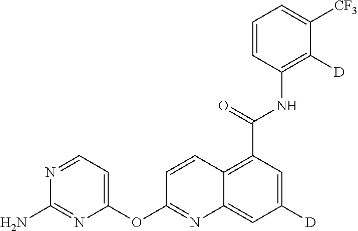 [SMILES: [2H]c1cc(C(=O)Nc2cccc(C(F)(F)F)c2[2H])c2ccc(Oc3ccnc(N)n3)nc2c1] |
| 2-8 | 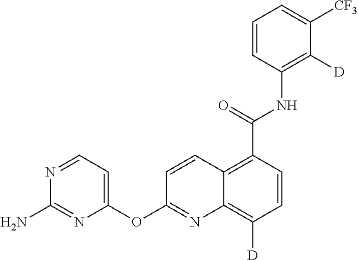 [SMILES: [2H]c1c(NC(=O)c2ccc([2H])c3nc(Oc4ccnc(N)n4)ccc23)cccc1C(F)(F)F] |
| 2-9 | 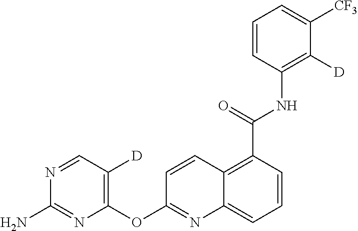 [SMILES: [2H]c1cnc(N)nc1Oc1ccc2c(C(=O)Nc3cccc(C(F)(F)F)c3[2H])cccc2n1] |
| 2-10 | 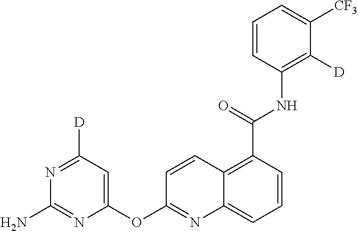 [SMILES: [2H]c1cc(Oc2ccc3c(C(=O)Nc4cccc(C(F)(F)F)c4[2H])cccc3n2)nc(N)n1] |
| 2-11 | 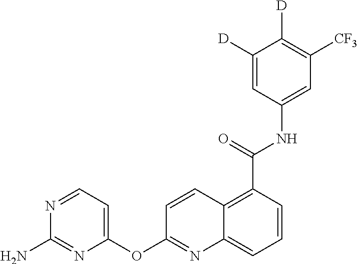 [SMILES: [2H]c1cc(NC(=O)c2cccc3nc(Oc4ccnc(N)n4)ccc23)cc(C(F)(F)F)c1[2H]] |
| 2-12 | 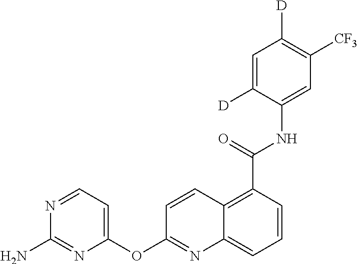 [SMILES: [2H]c1cc([2H])c(C(F)(F)F)cc1NC(=O)c1cccc2nc(Oc3ccnc(N)n3)ccc12] |
| 2-13 | 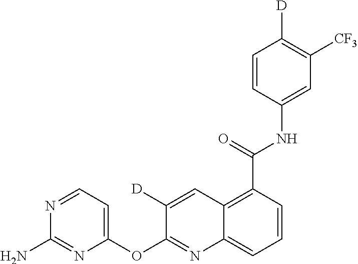 [SMILES: [2H]c1ccc(NC(=O)c2cccc3nc(Oc4ccnc(N)n4)c([2H])cc23)cc1C(F)(F)F] |
| 2-14 | 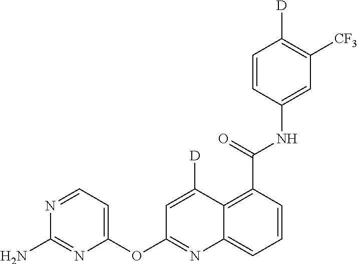 [SMILES: [2H]c1ccc(NC(=O)c2cccc3nc(Oc4ccnc(N)n4)cc([2H])c23)cc1C(F)(F)F] |
| 2-15 | 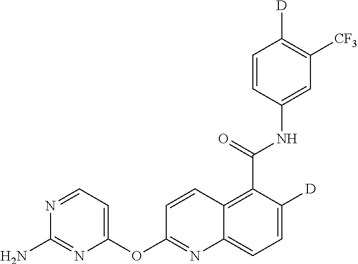 [SMILES: [2H]c1ccc(NC(=O)c2c([2H])ccc3nc(Oc4ccnc(N)n4)ccc23)cc1C(F)(F)F] |
| 2-16 | 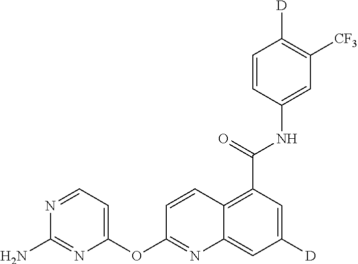 [SMILES: [2H]c1cc(C(=O)Nc2ccc([2H])c(C(F)(F)F)c2)c2ccc(Oc3ccnc(N)n3)nc2c1] |
| 2-17 | 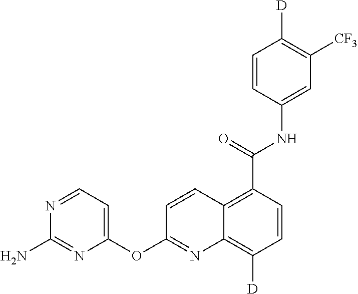 [SMILES: [2H]c1ccc(NC(=O)c2ccc([2H])c3nc(Oc4ccnc(N)n4)ccc23)cc1C(F)(F)F] |
| 2-18 | 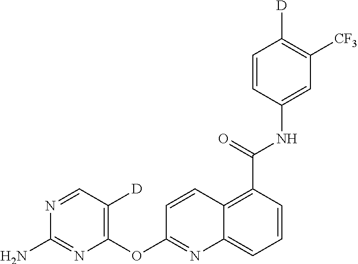 [SMILES: [2H]c1ccc(NC(=O)c2cccc3nc(Oc4nc(N)ncc4[2H])ccc23)cc1C(F)(F)F] |
| 2-19 | 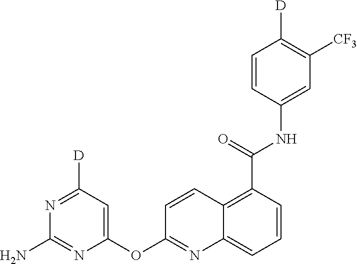 [SMILES: [2H]c1cc(Oc2ccc3c(C(=O)Nc4ccc([2H])c(C(F)(F)F)c4)cccc3n2)nc(N)n1] |
| 2-20 | 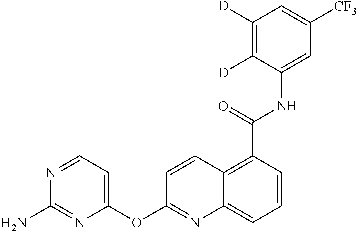 [SMILES: [2H]c1cc(C(F)(F)F)cc(NC(=O)c2cccc3nc(Oc4ccnc(N)n4)ccc23)c1[2H]] |
| 2-21 | 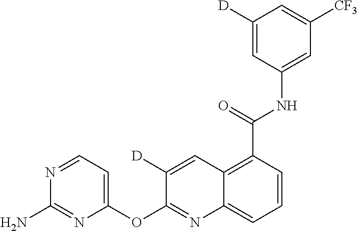 [SMILES: [2H]c1cc(NC(=O)c2cccc3nc(Oc4ccnc(N)n4)c([2H])cc23)cc(C(F)(F)F)c1] |
| 2-22 | 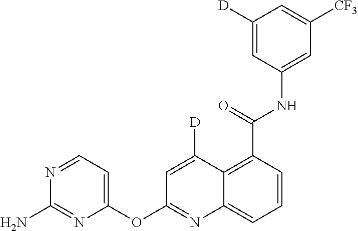 [SMILES: [2H]c1cc(NC(=O)c2cccc3nc(Oc4ccnc(N)n4)cc([2H])c23)cc(C(F)(F)F)c1] |
| 2-23 | 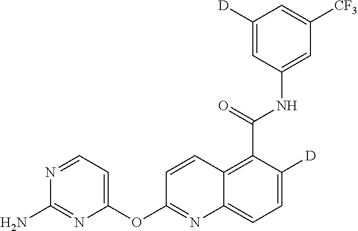 [SMILES: [2H]c1cc(NC(=O)c2c([2H])ccc3nc(Oc4ccnc(N)n4)ccc23)cc(C(F)(F)F)c1] |
| 2-24 | 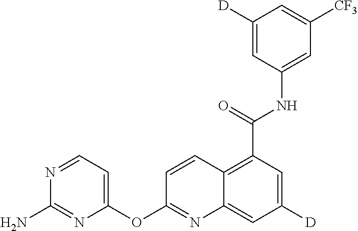 [SMILES: [2H]c1cc(NC(=O)c2cc([2H])cc3nc(Oc4ccnc(N)n4)ccc23)cc(C(F)(F)F)c1] |
| 2-25 | 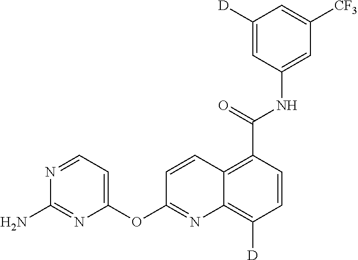 [SMILES: [2H]c1cc(NC(=O)c2ccc([2H])c3nc(Oc4ccnc(N)n4)ccc23)cc(C(F)(F)F)c1] |
| 2-26 | 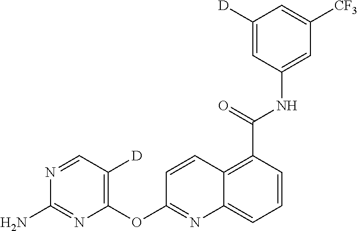 [SMILES: [2H]c1cc(NC(=O)c2cccc3nc(Oc4nc(N)ncc4[2H])ccc23)cc(C(F)(F)F)c1] |
| 2-27 | 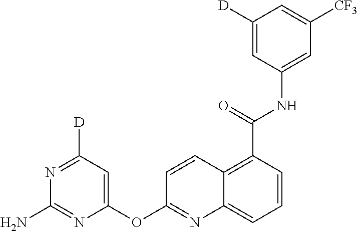 [SMILES: [2H]c1cc(NC(=O)c2cccc3nc(Oc4cc([2H])nc(N)n4)ccc23)cc(C(F)(F)F)c1] |
| 2-28 | 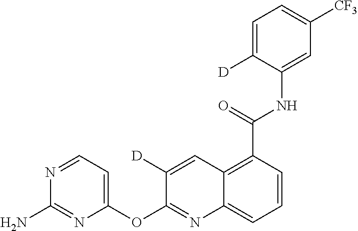 [SMILES: [2H]c1ccc(C(F)(F)F)cc1NC(=O)c1cccc2nc(Oc3ccnc(N)n3)c([2H])cc12] |
| 2-29 | 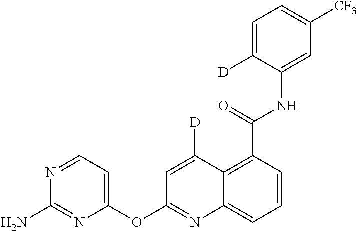 [SMILES: [2H]c1ccc(C(F)(F)F)cc1NC(=O)c1cccc2nc(Oc3ccnc(N)n3)cc([2H])c12] |
| 2-30 | 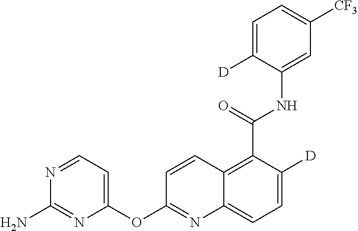 [SMILES: [2H]c1ccc(C(F)(F)F)cc1NC(=O)c1c([2H])ccc2nc(Oc3ccnc(N)n3)ccc12] |
| 2-31 | 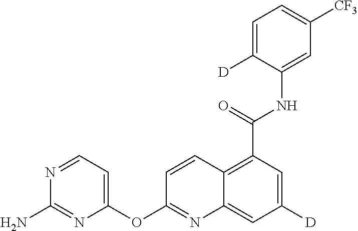 [SMILES: [2H]c1cc(C(=O)Nc2cc(C(F)(F)F)ccc2[2H])c2ccc(Oc3ccnc(N)n3)nc2c1] |
| 2-32 | 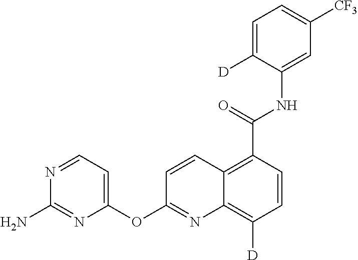 [SMILES: [2H]c1ccc(C(F)(F)F)cc1NC(=O)c1ccc([2H])c2nc(Oc3ccnc(N)n3)ccc12] |
| 2-33 | 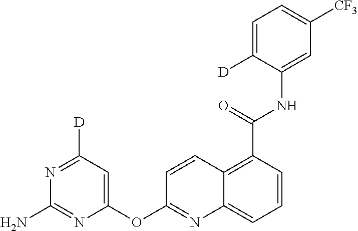 [SMILES: [2H]c1cc(Oc2ccc3c(C(=O)Nc4cc(C(F)(F)F)ccc4[2H])cccc3n2)nc(N)n1] |
| 2-34 | 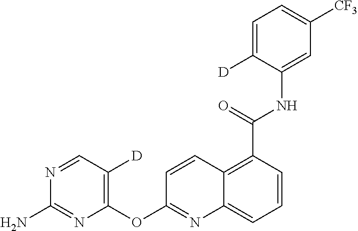 [SMILES: [2H]c1ccc(C(F)(F)F)cc1NC(=O)c1cccc2nc(Oc3nc(N)ncc3[2H])ccc12] |
| 2-35 | 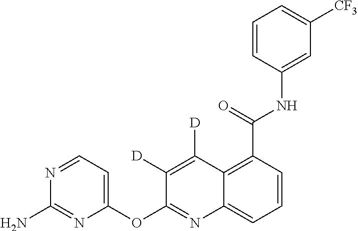 [SMILES: [2H]c1c(Oc2ccnc(N)n2)nc2cccc(C(=O)Nc3cccc(C(F)(F)F)c3)c2c1[2H]] |
| 2-36 | 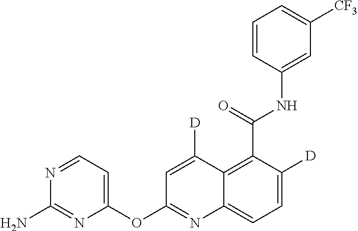 [SMILES: [2H]c1ccc2nc(Oc3ccnc(N)n3)cc([2H])c2c1C(=O)Nc1cccc(C(F)(F)F)c1] |
| 2-37 | 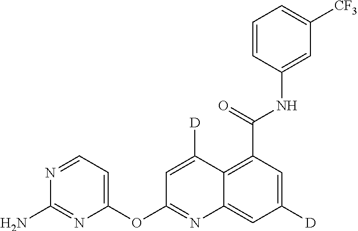 [SMILES: [2H]c1cc(C(=O)Nc2cccc(C(F)(F)F)c2)c2c([2H])cc(Oc3ccnc(N)n3)nc2c1] |
| 2-38 | 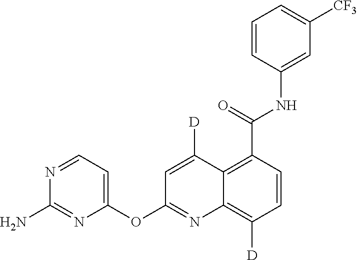 [SMILES: [2H]c1ccc(C(=O)Nc2cccc(C(F)(F)F)c2)c2c([2H])cc(Oc3ccnc(N)n3)nc12] |
| 2-39 | 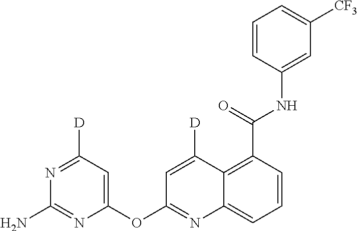 [SMILES: [2H]c1cc(Oc2cc([2H])c3c(C(=O)Nc4cccc(C(F)(F)F)c4)cccc3n2)nc(N)n1] |
| 2-40 | 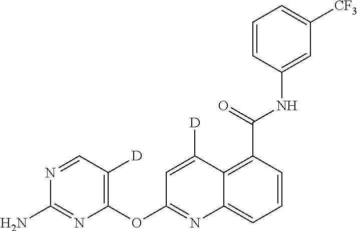 [SMILES: [2H]c1cnc(N)nc1Oc1cc([2H])c2c(C(=O)Nc3cccc(C(F)(F)F)c3)cccc2n1] |
| 2-41 | 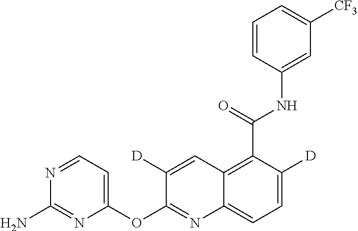 [SMILES: [2H]c1cc2c(C(=O)Nc3cccc(C(F)(F)F)c3)c([2H])ccc2nc1Oc1ccnc(N)n1] |
| 2-42 | 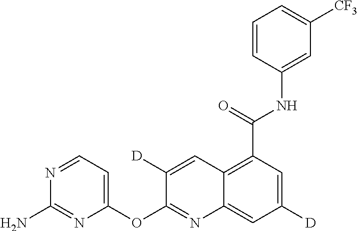 [SMILES: [2H]c1cc(C(=O)Nc2cccc(C(F)(F)F)c2)c2cc([2H])c(Oc3ccnc(N)n3)nc2c1] |
| 2-43 | 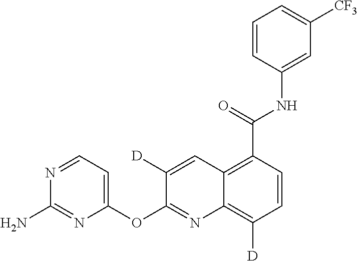 [SMILES: [2H]c1cc2c(C(=O)Nc3cccc(C(F)(F)F)c3)ccc([2H])c2nc1Oc1ccnc(N)n1] |
| 2-44 | 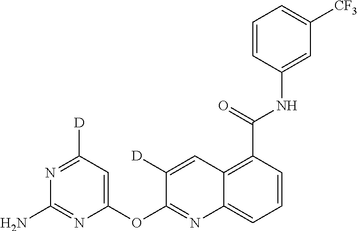 [SMILES: [2H]c1cc(Oc2nc3cccc(C(=O)Nc4cccc(C(F)(F)F)c4)c3cc2[2H])nc(N)n1] |
| 2-45 | 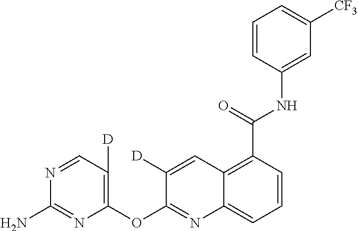 [SMILES: [2H]c1cnc(N)nc1Oc1nc2cccc(C(=O)Nc3cccc(C(F)(F)F)c3)c2cc1[2H]] |
| 2-46 | 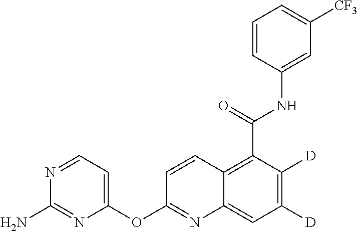 [SMILES: [2H]c1cc2nc(Oc3ccnc(N)n3)ccc2c(C(=O)Nc2cccc(C(F)(F)F)c2)c1[2H]] |
| 2-47 | 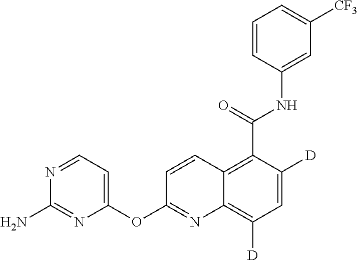 [SMILES: [2H]c1cc([2H])c2nc(Oc3ccnc(N)n3)ccc2c1C(=O)Nc1cccc(C(F)(F)F)c1] |
| 2-48 | 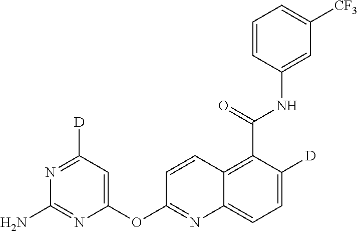 [SMILES: [2H]c1cc(Oc2ccc3c(C(=O)Nc4cccc(C(F)(F)F)c4)c([2H])ccc3n2)nc(N)n1] |
| 2-49 | 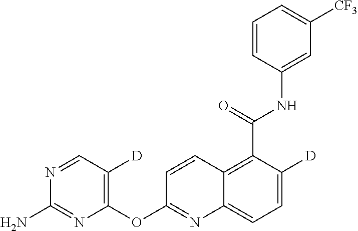 [SMILES: [2H]c1cnc(N)nc1Oc1ccc2c(C(=O)Nc3cccc(C(F)(F)F)c3)c([2H])ccc2n1] |
| 2-50 | 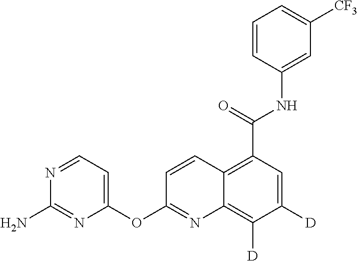 [SMILES: [2H]c1cc(C(=O)Nc2cccc(C(F)(F)F)c2)c2ccc(Oc3ccnc(N)n3)nc2c1[2H]] |
| 2-51 | 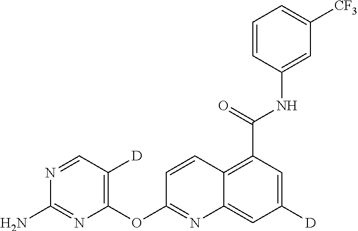 [SMILES: [2H]c1cc(C(=O)Nc2cccc(C(F)(F)F)c2)c2ccc(Oc3nc(N)ncc3[2H])nc2c1] |
| 2-52 | 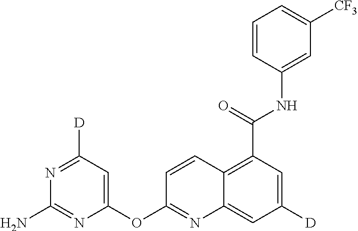 [SMILES: [2H]c1cc(C(=O)Nc2cccc(C(F)(F)F)c2)c2ccc(Oc3cc([2H])nc(N)n3)nc2c1] |
| 2-53 | 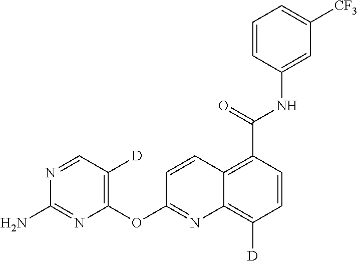 [SMILES: [2H]c1cnc(N)nc1Oc1ccc2c(C(=O)Nc3cccc(C(F)(F)F)c3)ccc([2H])c2n1] |
| 2-54 | 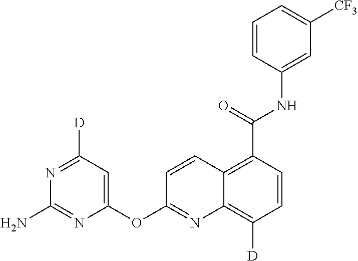 [SMILES: [2H]c1cc(Oc2ccc3c(C(=O)Nc4cccc(C(F)(F)F)c4)ccc([2H])c3n2)nc(N)n1] |
| 2-55 | 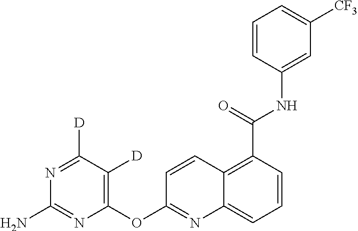 [SMILES: [2H]c1nc(N)nc(Oc2ccc3c(C(=O)Nc4cccc(C(F)(F)F)c4)cccc3n2)c1[2H]] |

In one aspect, provided herein is a compound of Formula (II), or a pharmaceutically acceptable salt thereof, or a stereoisomer thereof, or a hydrate or a solvate thereof:

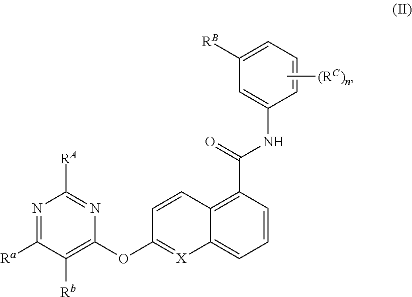

or a pharmaceutically acceptable salt thereof, wherein:
X is N or CH;
R^{A} is H, —N(R^{x})₂, —OR^{x}, —SR^{x}, —S(O)R^{x}, —S(O)₂R^{x}, or S(O)₂N(R^{x})₂ or C₁-C₆ alkyl optionally substituted with one or more independently selected —N(R^{x})₂ or —OR^{x};
R^{B} is C₁-C₆ alkyl optionally substituted with one or more halo;
R^{C} is H or —CR^{c}R^{d}R^{e}, wherein R^{c}, R^{d}, and R^{e} are each independently H, halogen, or C₁-C₆ alkyl optionally substituted with one or more independently selected halogen;
n is an integer from 0 to 4;
R^{a} is H, —N(R^{x})₂, —OR^{x}, —SR^{x}, —S(O)R^{x}, —S(O)₂R^{x}, S(O)₂N(R^{x})₂ or C₁-C₆ alkyl optionally substituted with one or more independently selected —N(R^{x})₂ or —OR;
R^{b} is H or C₁-C₆ alkyl;
or, R^{a} and R^{b} together with the carbon atom connecting them form a phenyl, C₅-C₆ cycloalkyl, 5- to 6-membered heteroaryl, or 5- to 6-membered heterocyclyl, each of which is optionally substituted by one or more independently selected halo or C₁-C₆ alkyl;
wherein R^{x}, at each occurrence, is independently H or C₁-C₆ alkyl; and
wherein one or more of the hydrogen(s) in Formula (II) is optionally replaced by deuterium.

In one aspect, provided herein is a compound of Formula (II), or a pharmaceutically acceptable salt thereof, or a stereoisomer thereof, or a hydrate or a solvate thereof:

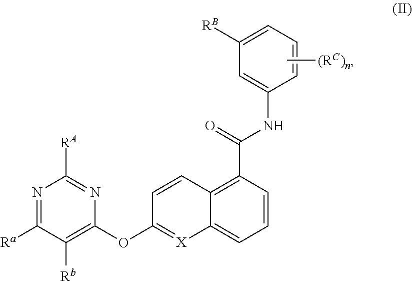

or a pharmaceutically acceptable salt thereof, wherein:
X is N or CH;
one of R^{A} and R^{a} is —N(R^{x})₂ and the other is H;
R^{B} is C₁-C₆ alkyl optionally substituted with one or more halo;
R^{C} is deuterium;
R^{b} is H or C₁-C₆ alkyl;
wherein RY, at each occurrence, is independently H or C₁-C₆ alkyl; and
wherein one or more of the hydrogen(s) in Formula (II) is optionally replaced by deuterium.

In one aspect, provided herein is a compound of Formula (II), or a pharmaceutically acceptable salt thereof, or a stereoisomer thereof, or a hydrate or a solvate thereof:

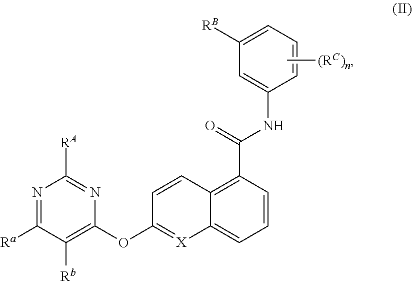

or a pharmaceutically acceptable salt thereof, wherein:
X is N or CH;
one of R^{A} and R^{a} is —NH₂ and the other is H;
R^{B} is C₁-C₆ alkyl optionally substituted with one or more halo;
R^{C} is deuterium;
R^{b} is H or C₁-C₆ alkyl;
wherein one or more of the hydrogen(s) in Formula (II) is optionally replaced by deuterium.

In one aspect, provided herein is a compound of Formula (II), or a pharmaceutically acceptable salt thereof, or a stereoisomer thereof, or a hydrate or a solvate thereof:

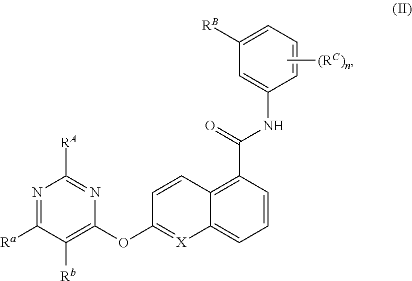

or a pharmaceutically acceptable salt thereof, wherein:
X is N or CH;
one of R^{A} and R^{a} is C₁-C₆ alkyl optionally substituted with one or more independently selected —N(R^{x})₂ or —OR^{x} and the other is H;
R^{B} is C₁-C₆ alkyl optionally substituted with one or more halo;
R^{C} is H or —CR^{c}R^{d}R^{e}, wherein R^{e}, R^{d}, and R^{e} are each independently H, halogen, or C₁-C₆ alkyl optionally substituted with one or more independently selected halogen;
n is an integer from 0 to 4;
R^{b} is H or C₁-C₆ alkyl;
wherein R^{x}, at each occurrence, is independently H or C₁-C₆ alkyl; and
wherein one or more of the hydrogen(s) in Formula (II) is optionally replaced by deuterium.

In one aspect, provided herein is a compound of Formula (II), or a pharmaceutically acceptable salt thereof, or a stereoisomer thereof, or a hydrate or a solvate thereof:

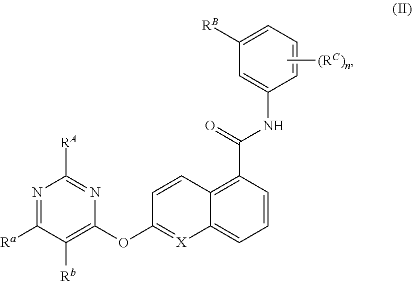

or a pharmaceutically acceptable salt thereof, wherein:
X is N or CH;
one of R^{A} and R^{a} is C₁-C₂ alkyl optionally substituted with one or more independently selected —N(R^{x})₂ or —OR^{x} and the other is H;
R^{B} is C₁-C₂ alkyl optionally substituted with one or more halo;
R^{C} is H or —CR^{c}R^{d}R^{e}, wherein R^{c}, R^{d}, and R^{e} are each independently H, halogen, or C₁-C₆ alkyl optionally substituted with one or more independently selected halogen;
n is an integer from 0 to 4;
R^{b} is H or C₁-C₆ alkyl;
wherein R^{x}, at each occurrence, is independently H or C₁-C₆ alkyl;
wherein one or more of the hydrogen(s) in Formula (II) is optionally replaced by deuterium.

In some embodiments, the compound is selected from the group consisting of:

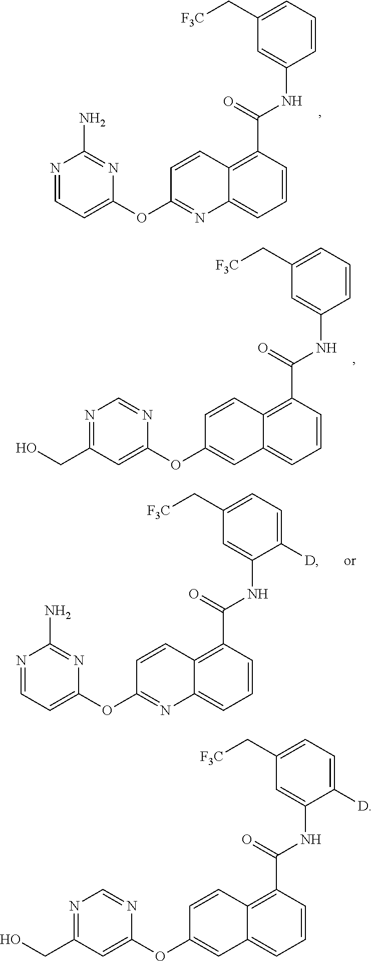

In some embodiments, the compound is

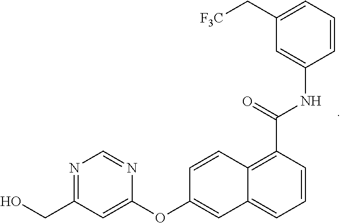 [SMILES: O=C(Nc1cccc(CC(F)(F)F)c1)c1cccc2cc(Oc3cc(CO)ncn3)ccc12]

In some embodiments, the compound is

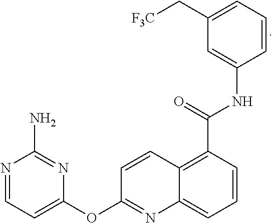 [SMILES: Nc1nccc(Oc2ccc3c(C(=O)Nc4cccc(CC(F)(F)F)c4)cccc3n2)n1]

In some embodiments, the disclosure also includes any or all of the stereochemical forms, including any enantiomeric or diastereomeric forms, and any tautomers or other forms of the compounds described. In some embodiments, unless stereochemistry is explicitly indicated in a chemical structure or name, the structure or name is intended to embrace all possible stereoisomers of a compound depicted. In some embodiments, where a specific stereochemical form is depicted, it is understood that other stereochemical forms are also described and embraced by the disclosure. All forms of the compounds are also embraced by the disclosure, such as crystalline or non-crystalline forms of the compounds. It is also understood that prodrugs, solvates and metabolites of the compounds are embraced by this disclosure. Compositions comprising a compound described herein are also intended, such as a composition of substantially pure compound, including a specific stereochemical form thereof. Compositions comprising a mixture of compounds described herein in any ratio are also embraced by the disclosure, including mixtures of two or more stereochemical forms of a compound in any ratio, such that racemic, non-racemic, enantioenriched and scalemic mixtures of a compound are embraced.

In some embodiments, the disclosure also includes further isotopically-labeled and/or isotopically-enriched forms of compounds described herein. The compounds herein may contain unnatural proportions of atomic isotopes in addition to deuterium at one or more of the atoms that constitute such compounds. Exemplary additional isotopes that can be further incorporated into compounds described herein include isotopes of hydrogen, carbon, nitrogen, oxygen, sulfur, chlorine, such as ³H, ¹¹C, ¹³C, ¹⁴C ¹³N, ¹⁵O, ¹⁷O, ³⁵S, ¹⁸F, ³⁶Cl. As used herein, each instance of enrichment, substitution, or replacement of an atom with corresponding isotope of that atom encompasses isotopic enrichment levels of one of about: 50%, 60%, 70%, 80%, 90%, 95%, 96%, 97%, 98%, 99%, 99.5%, 99.6%, 99.7%, 99.8%, 99.9%, or 100%, or a range between any two of the preceding percentages.

### III. Method of Enhancing Anti-VEGFR Activity

In one aspect, provided herein is a method of improving the efficacy of an VEGFR2 protein kinase inhibitor in inhibiting VEGFR2 protein kinase, comprising substituting at least one hydrogen atom of the inhibitor with deuterium. In some embodiments, provided herein is a method of improving the efficacy of compound 2-2 in inhibiting VEGFR2 protein kinase

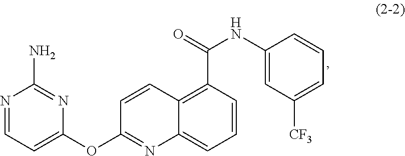 [SMILES: Nc1nccc(Oc2ccc3c(C(=O)Nc4cccc(C(F)(F)F)c4)cccc3n2)n1]

comprising substituting at least one hydrogen in compound 2-2 with deuterium. In some embodiments, the hydrogen in compound 2-2 is a hydrogen connected to an aromatic ring or aromatic bicyclic ring. In some embodiments, the hydrogen is a hydrogen on the phenyl. In some embodiments, the hydrogen is a hydrogen on the quinoline. In some embodiments, the hydrogen is a hydrogen on the pyrimidine. In some embodiments, the method comprises substituting at least about 50% (e.g., at least about any of 60%, 70%, 80%, 90%, 95%, 96%, 97%, 98%, 99%, 99.5%, 99.6%, 99.7%, 99.8%, 99.9%, or 100%) of the hydrogen at a position of compound 2-2 with deuterium. In some embodiments, the method comprises substituting at least two hydrogen (e.g., any of 2, 3, 4, 5, 6, 7, 8, 9, 10, or 11) in compound 2-2 with deuterium.

### IV. Pharmaceutically Acceptable Salt and Composition

In some embodiments, provided herein is a salt form of the compounds (e.g., compound of Formula (I), compound selected from Table 1, or compound selected from Table 2), described herein, for example, a pharmaceutically acceptable salt.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have in some embodiments met the required standards of toxicological and manufacturing testing and/or are included on the Inactive Ingredient Guide prepared by the U.S. Food and Drug administration.

In some embodiments, the salt (e.g., pharmaceutically acceptable salts) of the compounds provided herein retain at least some of the biological activity of the free (non-salt) compound and can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound in its free acid or base form with a suitable organic or inorganic base or acid, respectively, and isolating the salt thus formed during subsequent purification.

In some embodiments, suitable inorganic acids include, but not limited to, haloid acid (e.g., hydrochloric acid), sulfuric acid, or phosphoric acid. In some embodiments, suitable organic acids include, but not limited to, carboxylic acid, phosphoric acid, sulfonic acid or aminocarboxylic acid, for example, acetic acid, propionic acid, octanoic acid, decanoic acid, dodecanoic acid, hydroxyacetic acid, lactic acid, fumaric acid, succinic acid, adipic acid, pimelic acid, suberic acid, azelaic acid, malic acid, tartaric acid, citric acid, amino acid, e.g., glutamic acid or aspartic acid, maleic acid, hydroxy acid, methyl maleic acid, cyclohexanecarboxylic acid, adamantanecarboxylic acid, benzoic acid, salicylic acid, 4-amino salicylic acid, phthalic acid, phenylacetic acid, mandelic acid, cinnamic acid, methane or ethane sulfonic acid, 2-oxyethylsulfonic acid, ethane-1,2-disulfonic acid, phenylsulfonic acid, 2-naphthalene sulfonic acid, 1,5-naphthalene disulfonic acid, 2-toluene sulfonic acid, p-toluene sulfonic acid, ethylsulfuric acid, dodecyl sulfuric acid, N-cyclohexyl amino acetic acid, N-methyl-N-ethyl-N-propyl-sulfamic acid, or other organic acids, e.g., ascorbic acid.

In some embodiments, the salt (e.g., pharmaceutically acceptable salts) of the compounds described herein comprise phosphate salt, D-camphorsulfonate salt, chloride salt, bromide salt, fluoride salt, sulfate salt, nitrate salt, formate salt, acetate salt, propionate salt, oxalate salt, malonate salt, succinate salt, fumarate salt, maleate salt, lactate salt, malate salt, tartrate salt, citrate salt, picrate salt, methanesulfonate salt, benzene mesylate salt, benzenesulfonate salt, aspartate salt, or glutamate salt.

In another aspect, provided herein is a composition (e.g., pharmaceutical composition) comprising a compound described herein, or a pharmaceutically acceptable salt thereof, or a stereoisomer thereof, or a hydrate or a solvate thereof. In one embodiment, the composition is an ophthalmic formulation. In some embodiments, the ophthalmic preparation can further comprise other known medicaments having similar therapeutic use. In some embodiments, the ophthalmic formulation is an eye drop, an eye ointment or an ophthalmic injectable composition. In some embodiments, the ophthalmic injectable composition is suitable for intravitreal or subconjunctival injectable composition.

### V. Methods of Treatment

In some embodiments, provided herein is use of the above compounds (e.g., compound of Formula (I), compound selected from Table 1, or compound selected from Table 2), or a pharmaceutically acceptable salt thereof, or a stereoisomer thereof, or a hydrate or a solvate thereof, or a composition thereof, in preparation of a medicament for inhibiting abnormal angiogenic cell proliferation or angiogenesis or treating a disease or condition associated with abnormal angiogenic cell proliferation or angiogenesis. In some embodiments, the medicament for inhibiting the abnormal angiogenic cell proliferation or angiogenesis is vascular endothelial cell growth factor receptor 2 (VEGFR2) inhibitor. In some embodiments, the medicament for inhibiting the abnormal angiogenic cell proliferation or angiogenesis is a medicament against ocular angiogenesis. In some embodiments, the medicament for inhibiting the abnormal angiogenic cell proliferation or angiogenesis is choroidal angiogenesis inhibitor. In some embodiments, the medicament for inhibiting the abnormal angiogenic cell proliferation or angiogenesis is a medicament for treating or preventing wet macular degeneration, diabetic retinopathy or neovascular glaucoma.

In some embodiments, the medicament is an ophthalmic preparation. In some embodiments, the ophthalmic preparation is an eye drop, an eye ointment or an ophthalmic injection. In some embodiments, the ophthalmic injection is an intravitreal injection.

In some embodiments, provided herein is use of the above compounds, or a pharmaceutically acceptable salt thereof, or a stereoisomer thereof, or a hydrate or a solvate thereof, or a composition thereof, in preparation of a medicament for treating diseases associated with the abnormal angiogenic cell proliferation or angiogenesis.

In some embodiments, the diseases associated with the abnormal angiogenic cell proliferation or angiogenesis are diseases caused by the abnormity of vascular endothelial cell growth factor receptor 2 (VEGFR2). In some embodiments, the diseases associated with the abnormal angiogenic cell proliferation or angiogenesis are diseases associated with ocular angiogenesis. In some embodiments, the diseases associated with the abnormal angiogenic cell proliferation or angiogenesis are diseases associated with choroidal angiogenesis. In some embodiments, the diseases associated with the abnormal angiogenic cell proliferation or angiogenesis are diseases associated with wet macular degeneration, diabetic retinopathy or neovascular glaucoma.

In another aspect, provided herein is a method of inhibiting the abnormal angiogenic cell proliferation or angiogenesis or treating diseases associated with the abnormal angiogenic cell proliferation or angiogenesis, comprising administering an effective amount (e.g., therapeutically effective amount) of compound of the present invention, or a pharmaceutically acceptable salt thereof, or a stereoisomer thereof, or a hydrate or a solvate thereof, or a composition thereof, to a subject in need thereof.

In some embodiments, the inhibition of the abnormal angiogenic cell proliferation or angiogenesis is inhibiting the abnormal proliferation of ocular angiogenesis; and the diseases associated with the abnormal angiogenic cell proliferation or angiogenesis are diseases associated with ocular angiogenesis.

In some embodiments, the inhibition of the abnormal angiogenic cell proliferation or angiogenesis is inhibiting the abnormal proliferation of choroidal angiogenesis; and the diseases associated with the abnormal angiogenic cell proliferation or angiogenesis are diseases associated with choroidal angiogenesis.

In some embodiments, the method of inhibiting the abnormal angiogenic cell proliferation or angiogenesis or treating diseases associated with the abnormal angiogenic cell proliferation or angiogenesis specifically is a method of treating or preventing wet macular degeneration, diabetic retinopathy or neovascular glaucoma.

In some embodiments, the administration is a topical administration direct to the ocular region or intravitreal or subconjunctival injection.

In yet another aspect, provided herein is use of the above compound, or a pharmaceutically acceptable salt thereof, or a stereoisomer thereof, or a hydrate or a solvate thereof, or a composition thereof, in the preparation of a protein kinase inhibitor medicament.

In yet another aspect, provided herein is use of the above compound, or a pharmaceutically acceptable salt thereof, or a stereoisomer thereof, or a hydrate or a solvate thereof, or a composition thereof, in the preparation of a medicament for treating diseases caused by the abnormal protein kinases.

In yet another aspect, provided herein is a method of treating diseases caused by the abnormal protein kinases, comprising administering an effective amount (e.g., therapeutically effective amount) of compound of the present invention, or a pharmaceutically acceptable salt thereof, or a stereoisomer thereof, or a hydrate or a solvate thereof, or a composition thereof, to a subject in need thereof. In some embodiments, the protein kinases comprises VEGFR2, PDGFR-β, KIT, AURORA-B, FGFR2, SRC, JAK2 or P38-α, preferably is VEGFR2, KIT or PDGFR-β, or any combination thereof.

In some embodiments, the diseases caused by the abnormal protein kinases comprise inflammation, neoplasm, malignancy, or tumor, or a combination thereof.

In some embodiments, provided herein is a method of treating a disease or condition associated with ocular angiogenesis in a subject in need thereof, comprising administering an effective amount of a compound provided herein, or a pharmaceutically acceptable salt thereof, or a stereoisomer thereof, or a hydrate or a solvate thereof, or a pharmaceutically composition comprising the compound, to the subject. In some embodiments, provided herein is a method of treating a disease or condition associated with choroidal angiogenesis in a subject in need thereof, comprising administering an effective amount of a compound provided herein, or a pharmaceutically acceptable salt thereof, or a stereoisomer thereof, or a hydrate or a solvate thereof, or a pharmaceutically composition comprising the compound, to the subject. In some embodiments, provided herein is a method of treating wet macular degeneration, diabetic retinopathy, or neovascular glaucoma in a subject in need thereof, comprising administering an effective amount of a compound provided herein, or a pharmaceutically acceptable salt thereof, or a stereoisomer thereof, or a hydrate or a solvate thereof, or a pharmaceutically composition comprising the compound, to the subject. In some embodiments, the subject has not been treated with compound

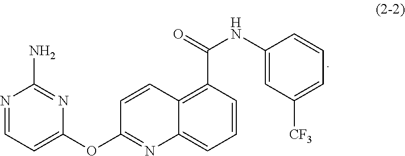 [SMILES: Nc1nccc(Oc2ccc3c(C(=O)Nc4cccc(C(F)(F)F)c4)cccc3n2)n1]

In some embodiments, the subject has been treated with compound

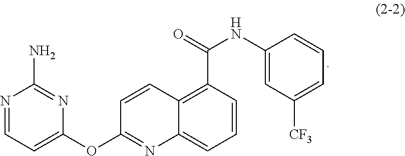 [SMILES: Nc1nccc(Oc2ccc3c(C(=O)Nc4cccc(C(F)(F)F)c4)cccc3n2)n1]

In some embodiments, the subject is more responsive to a compound provided herein, or a pharmaceutically acceptable salt thereof, or a stereoisomer thereof, or a hydrate or a solvate thereof, or a pharmaceutically composition provided herein, compared to compound

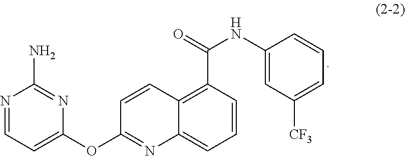 [SMILES: Nc1nccc(Oc2ccc3c(C(=O)Nc4cccc(C(F)(F)F)c4)cccc3n2)n1]

In some embodiments, the subject is at least or about 1.5 times, at least or about 2 times, at least or about 2.5 times, at least or about 3 times, or more responsive to the compound provided herein compared to compound

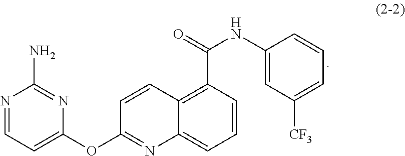 [SMILES: Nc1nccc(Oc2ccc3c(C(=O)Nc4cccc(C(F)(F)F)c4)cccc3n2)n1]

Exemplary Embodiments of the present disclosure include:
Embodiment 1: A compound of structure

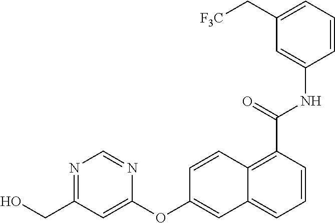 [SMILES: O=C(Nc1cccc(CC(F)(F)F)c1)c1cccc2cc(Oc3cc(CO)ncn3)ccc12]

or of Formula (I), or a pharmaceutically acceptable salt thereof, or a stereoisomer thereof, or a hydrate or a solvate thereof:

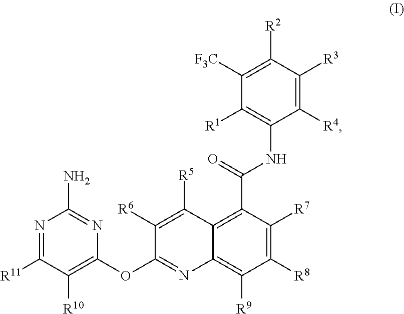

wherein R¹, R², R³, R⁴, R⁵, R⁶, R⁷, R⁸, R⁹, R¹⁰, and R¹¹, are each independently hydrogen or deuterium, and at least one of R¹, R², R³, R⁴, R⁵, R⁶, R⁷, R⁸, R⁹, R¹⁰, and R¹¹ is deuterium.
Embodiment 2: The compound of Embodiment 1, or a pharmaceutically acceptable salt thereof, or a stereoisomer thereof, or a hydrate or a solvate thereof, wherein R¹ is deuterium, and optionally wherein 1, 2, 3, 4, 5, 6, 7, 8, 9, or 10 of R², R³, R⁴, R⁵, R⁶, R⁷, R⁸, R⁹, R¹⁰, and R¹¹ is deuterium.
Embodiment 3: The compound of Embodiment 1, or a pharmaceutically acceptable salt thereof, or a stereoisomer thereof, or a hydrate or a solvate thereof, wherein R² is deuterium, and optionally wherein 1, 2, 3, 4, 5, 6, 7, 8, 9, or 10 of R¹, R³, R⁴, R⁵, R⁶, R⁷, R⁸, R⁹, R¹⁰, and R¹¹ is deuterium.
Embodiment 4: The compound of Embodiment 1, or a pharmaceutically acceptable salt thereof, or a stereoisomer thereof, or a hydrate or a solvate thereof, wherein R³ is deuterium, and optionally wherein 1, 2, 3, 4, 5, 6, 7, 8, 9, or 10 of R¹, R², R⁴, R⁵, R⁶, R⁷, R⁸, R⁹, R¹⁰, and R¹¹ is deuterium.
Embodiment 5: The compound of Embodiment 1, or a pharmaceutically acceptable salt thereof, or a stereoisomer thereof, or a hydrate or a solvate thereof, wherein R⁴ is deuterium, and optionally wherein 1, 2, 3, 4, 5, 6, 7, 8, 9, or 10 of R¹, R², R³, R⁵, R⁶, R⁷, R⁸, R⁹, R¹⁰, and R¹¹ is deuterium.
Embodiment 6: The compound of Embodiment 1, or a pharmaceutically acceptable salt thereof, or a stereoisomer thereof, or a hydrate or a solvate thereof, wherein R⁵ is deuterium, and optionally wherein 1, 2, 3, 4, 5, 6, 7, 8, 9, or 10 of R¹, R², R³, R⁴, R⁶, R⁷, R⁸, R⁹, R¹⁰, and R¹¹ is deuterium.
Embodiment 7: The compound of Embodiment 1, or a pharmaceutically acceptable salt thereof, or a stereoisomer thereof, or a hydrate or a solvate thereof, wherein R⁶ is deuterium, and optionally wherein 1, 2, 3, 4, 5, 6, 7, 8, 9, or 10 of R¹, R², R³, R⁴, R⁵, R⁷, R⁸, R⁹, R¹⁰, and R¹¹ is deuterium.
Embodiment 8: The compound of Embodiment 1, or a pharmaceutically acceptable salt thereof, or a stereoisomer thereof, or a hydrate or a solvate thereof, wherein R⁷ is deuterium, and optionally wherein 1, 2, 3, 4, 5, 6, 7, 8, 9, or 10 of R¹, R², R³, R⁴, R⁵, R⁶, R⁸, R⁹, R¹⁰, and R¹¹ is deuterium.
Embodiment 9: The compound of Embodiment 1, or a pharmaceutically acceptable salt thereof, or a stereoisomer thereof, or a hydrate or a solvate thereof, wherein R⁸ is deuterium, and optionally wherein 1, 2, 3, 4, 5, 6, 7, 8, 9, or 10 of R¹, R², R³, R⁴, R⁵, R⁶, R⁷, R⁹, R¹⁰, and R¹¹ is deuterium.
Embodiment 10: The compound of Embodiment 1, or a pharmaceutically acceptable salt thereof, or a stereoisomer thereof, or a hydrate or a solvate thereof, wherein R⁹ is deuterium, and optionally wherein 1, 2, 3, 4, 5, 6, 7, 8, 9, or 10 of R¹, R², R³, R⁴, R⁵, R⁶, R⁷, R⁸, R¹⁰, and R¹¹ is deuterium.
Embodiment 11: The compound of Embodiment 1, or a pharmaceutically acceptable salt thereof, or a stereoisomer thereof, or a hydrate or a solvate thereof, wherein R¹⁰ is deuterium, and optionally wherein 1, 2, 3, 4, 5, 6, 7, 8, 9, or 10 of R¹, R², R³, R⁴, R⁵, R⁶, R⁷, R⁸, R⁹, and R¹¹ is deuterium.
Embodiment 12: The compound of Embodiment 1, or a pharmaceutically acceptable salt thereof, or a stereoisomer thereof, or a hydrate or a solvate thereof, wherein R¹¹ is deuterium, and optionally wherein 1, 2, 3, 4, 5, 6, 7, 8, 9, or 10 of R¹, R², R³, R⁴, R⁵, R⁶, R⁷, R⁸, R⁹, and R¹⁰ is deuterium.
Embodiment 13: The compound of Embodiment 1, or a pharmaceutically acceptable salt thereof, or a stereoisomer thereof, or a hydrate or a solvate thereof, selected from the group consisting of:

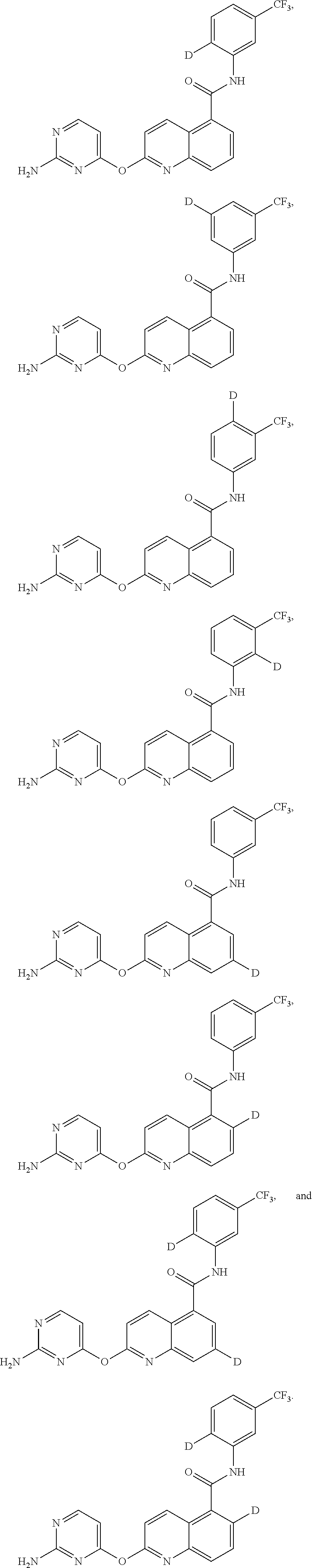

Embodiment 14: The compound of Embodiment 1, or a pharmaceutically acceptable salt thereof, or a stereoisomer thereof, or a hydrate or a solvate thereof, wherein the compound is

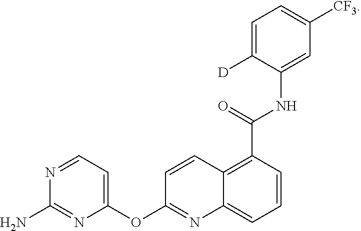 [SMILES: [2H]c1ccc(C(F)(F)F)cc1NC(=O)c1cccc2nc(Oc3ccnc(N)n3)ccc12]

Embodiment 15: A compound of Formula (II), or a pharmaceutically acceptable salt thereof, or a stereoisomer thereof, or a hydrate or a solvate thereof:

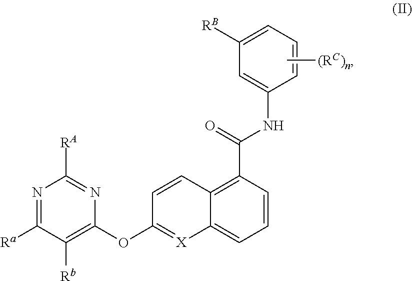

or a pharmaceutically acceptable salt thereof, wherein: X is N or CH; R^{A} is H, —N(R^{x})₂, —OR^{x}, —SR^{x}, —S(O)R^{x}, —S(O)₂R^{x}, or S(O)₂N(R^{x})₂ or C₁-C₆ alkyl optionally substituted with one or more independently selected —N(R^{x})₂ or —OR^{x}; R^{B} is C₁-C₆ alkyl optionally substituted with one or more halo; R^{C} is H or —CR^{c}R^{d}R^{e}, wherein R^{e}, R^{d}, and R^{e} are each independently H, halogen, or C₁-C₆ alkyl optionally substituted with one or more independently selected halogen; n is an integer from 0 to 4; R^{a} is H, —N(R^{x})₂, —OR^{x}, —SR^{x}, —S(O)R^{x}, —S(O)₂R^{x}, S(O)₂N(R^{x})₂ or C₁-C₆ alkyl optionally substituted with one or more independently selected —N(R^{x})₂ or —OR^{x}; Rbis H or C₁-C₆ alkyl; or, R^{a} and R^{b} together with the carbon atom connecting them form a phenyl, C₅-C₆ cycloalkyl, 5- to 6-membered heteroaryl, or 5- to 6-membered heterocyclyl, each of which is optionally substituted by one or more independently selected halo or C₁-C₆ alkyl; wherein R^{x}, at each occurrence, is independently H or C₁-C₆ alkyl; and wherein one or more of the hydrogen(s) in Formula (II) is optionally replaced by deuterium.
Embodiment 16: The compound of Embodiment 15, or a pharmaceutically acceptable salt thereof, or a stereoisomer thereof, or a hydrate or a solvate thereof, wherein X is N.
Embodiment 17: The compound of Embodiment 15, or a pharmaceutically acceptable salt thereof, or a stereoisomer thereof, or a hydrate or a solvate thereof, wherein X is CH.
Embodiment 18: The compound of any one of Embodiments 15-17, or a pharmaceutically acceptable salt thereof, or a stereoisomer thereof, or a hydrate or a solvate thereof, wherein R^{A} is H or —NH₂.
Embodiment 19: The compound of any one of Embodiments 15-18, or a pharmaceutically acceptable salt thereof, or a stereoisomer thereof, or a hydrate or a solvate thereof, wherein R^{A} is H.
Embodiment 20: The compound of any one of Embodiments 15-19, or a pharmaceutically acceptable salt thereof, or a stereoisomer thereof, or a hydrate or a solvate thereof, wherein R^{B} is C₂-C₆ alkyl optionally substituted with one or more F.
Embodiment 21: The compound of any one of Embodiments 15-20, or a pharmaceutically acceptable salt thereof, or a stereoisomer thereof, or a hydrate or a solvate thereof, wherein R^{B} is C₂-C₆ alkyl substituted with three F.
Embodiment 22: The compound of any one of Embodiments 15-21, or a pharmaceutically acceptable salt thereof, or a stereoisomer thereof, or a hydrate or a solvate thereof, wherein R^{B} is ethyl substituted three F.
Embodiment 23: The compound of any one of Embodiments 15-22, or a pharmaceutically acceptable salt thereof, or a stereoisomer thereof, or a hydrate or a solvate thereof, wherein R^{B} is —CH₂CF₃.
Embodiment 24: The compound of any one of Embodiments 15-23, or a pharmaceutically acceptable salt thereof, or a stereoisomer thereof, or a hydrate or a solvate thereof, wherein R^{a} is H.
Embodiment 25: The compound of any one of Embodiments 15-23, or a pharmaceutically acceptable salt thereof, or a stereoisomer thereof, or a hydrate or a solvate thereof, wherein R^{a} is C₁-C₆ alkyl optionally substituted with one or more independently selected —N(R^{x})₂ or —OR^{x}.
Embodiment 26: The compound of any one of Embodiments 15-23 and 25, or a pharmaceutically acceptable salt thereof, or a stereoisomer thereof, or a hydrate or a solvate thereof, wherein R^{a} is C₁-C₆ alkyl optionally substituted with one or more-OH.
Embodiment 27: The compound of any one of Embodiments 15-23, 25, and 26, or a pharmaceutically acceptable salt thereof, or a stereoisomer thereof, or a hydrate or a solvate thereof, wherein R^{a} is —CH₂OH.
Embodiment 28: The compound of any one of Embodiments 15-27, or a pharmaceutically acceptable salt thereof, or a stereoisomer thereof, or a hydrate or a solvate thereof, wherein n is 0.
Embodiment 29: The compound of any one of Embodiments 15-28, or a pharmaceutically acceptable salt thereof, or a stereoisomer thereof, or a hydrate or a solvate thereof, wherein at least one hydrogen in Formula (II) is replaced by D.
Embodiment 30: The compound of any one of Embodiments 15-29, or a pharmaceutically acceptable salt thereof, or a stereoisomer thereof, or a hydrate or a solvate thereof, wherein the compound is of Formula (II-1):

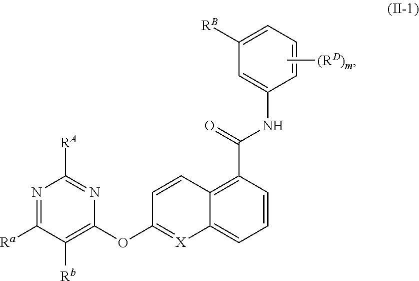

wherein R^{D} is D, and m is an integer from 0 to 4.
Embodiment 31: The compound of any one of Embodiments 15-30, or a pharmaceutically acceptable salt thereof, or a stereoisomer thereof, or a hydrate or a solvate thereof, wherein the compound is of Formula (II-2):

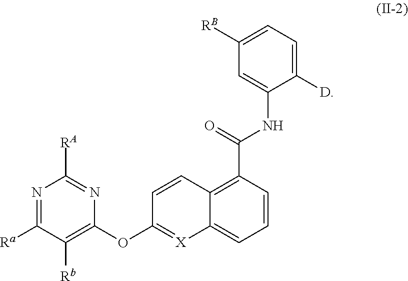

Embodiment 32: The compound of any one of Embodiments 15-31, or a pharmaceutically acceptable salt thereof, or a stereoisomer thereof, or a hydrate or a solvate thereof, wherein the compound is of Formula (II-a):

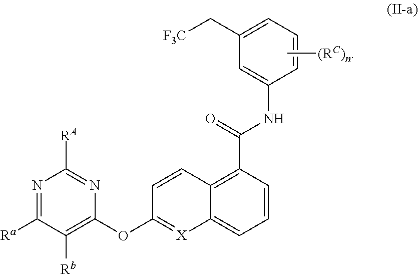

Embodiment 33: The compound of any one of Embodiments 15-32, or a pharmaceutically acceptable salt thereof, or a stereoisomer thereof, or a hydrate or a solvate thereof, wherein the compound is of Formula (II-b):

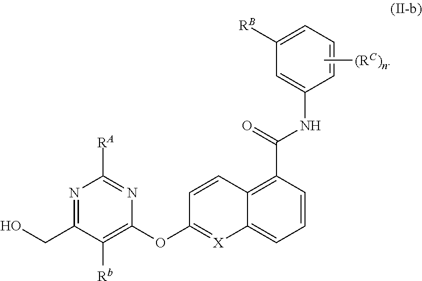

Embodiment 34: The compound of any one of Embodiments 15-33, or a pharmaceutically acceptable salt thereof, or a stereoisomer thereof, or a hydrate or a solvate thereof, wherein the compound is of Formula (II-2a):

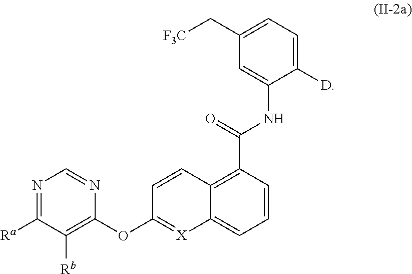

Embodiment 35: The compound of any one of Embodiments 15-34, or a pharmaceutically acceptable salt thereof, or a stereoisomer thereof, or a hydrate or a solvate thereof, wherein R^{b} is H.
Embodiment 36: The compound of Embodiment 15, or a pharmaceutically acceptable salt thereof, or a stereoisomer thereof, or a hydrate or a solvate thereof, wherein the compound is:

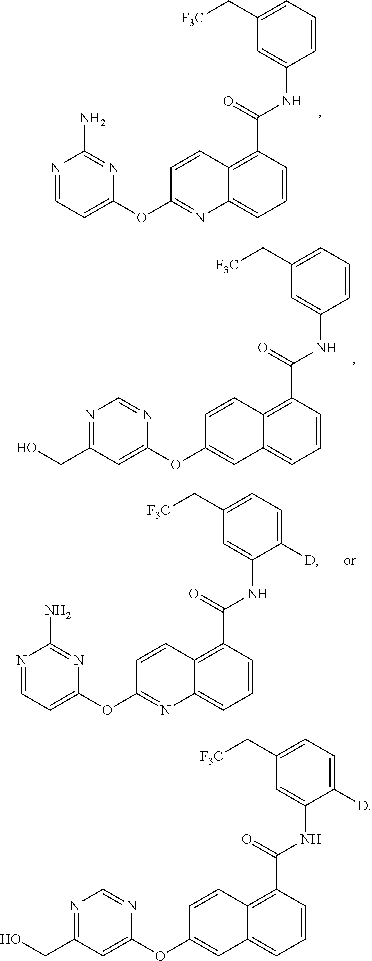

Embodiment 37: The compound of any one of Embodiments 1-36, or a pharmaceutically acceptable salt thereof, or a stereoisomer thereof, or a hydrate or a solvate thereof, wherein the pharmaceutically acceptable salt is selected from the group consisting of phosphate salt, D-camphorsulfonate salt, chloride salt, bromide salt, fluoride salt, sulfate salt, nitrate salt, formate salt, acetate salt, propionate salt, oxalate salt, malonate salt, succinate salt, fumarate salt, maleate salt, lactate salt, malate salt, tartrate salt, citrate salt, picrate salt, methanesulfonate salt, benzene mesylate salt, benzenesulfonate salt, aspartate salt, and glutamate salt.
Embodiment 38: A pharmaceutical composition comprises the compound of any one of Embodiments 1-36, or a pharmaceutically acceptable salt thereof, or a stereoisomer thereof, or a hydrate or a solvate thereof.
Embodiment 39: The pharmaceutical composition of Embodiment 38, being an ophthalmic formulation.
Embodiment 40: The pharmaceutical composition of any one of Embodiments 38 or 39, in the form of eye drop, eye ointment or ophthalmic injectable composition.
Embodiment 41: Use of the compound of any one of Embodiments 1-37, or a pharmaceutically acceptable salt thereof, or a stereoisomer thereof, or a hydrate or a solvate thereof, or the pharmaceutically composition of any one of Embodiments 38-40, in the preparation of a medicament for inhibiting abnormal angiogenic cell proliferation or angiogenesis or treating a disease or condition associated with abnormal angiogenic cell proliferation or angiogenesis.
Embodiment 42: The use of Embodiment 41, wherein the medicament for inhibiting abnormal angiogenic cell proliferation or angiogenesis is a VEGFR2 inhibitor.
Embodiment 43: The use of Embodiment 41 or 42, wherein the medicament for inhibiting abnormal angiogenic cell proliferation or angiogenesis is a medicament against ocular angiogenesis and the disease or condition associated with abnormal angiogenic cell proliferation or angiogenesis is a disease or condition associated with ocular angiogenesis.
Embodiment 44: The use of Embodiment 41 or 42, wherein the medicament for inhibiting abnormal angiogenic cell proliferation or angiogenesis is a choroidal angiogenesis inhibitor and the disease or condition associated with abnormal angiogenic cell proliferation or angiogenesis is a disease or condition associated with choroidal angiogenesis.
Embodiment 45: The use of any one of Embodiments 41-44, wherein the medicament for inhibiting abnormal angiogenic cell proliferation or angiogenesis is a medicament for treating wet macular degeneration, diabetic retinopathy, or neovascular glaucoma, and wherein the disease is wet macular degeneration, diabetic retinopathy, or neovascular glaucoma.
Embodiment 46: The compound of any one of Embodiments 1-37, or a pharmaceutically acceptable salt thereof, or a stereoisomer thereof, or a hydrate or a solvate thereof, or the pharmaceutically composition of any one of Embodiments 38-40, for inhibiting abnormal angiogenic cell proliferation or angiogenesis or treating a disease or condition associated with abnormal angiogenic cell proliferation or angiogenesis.
Embodiment 47: The compound of any one of Embodiments 1-37, or a pharmaceutically acceptable salt thereof, or a stereoisomer thereof, or a hydrate or a solvate thereof, or the pharmaceutically composition of any one of Embodiments 38-40, for inhibiting ocular angiogenesis.
Embodiment 48: The compound of any one of Embodiments 1-37, or a pharmaceutically acceptable salt thereof, or a stereoisomer thereof, or a hydrate or a solvate thereof, or the pharmaceutically composition of any one of Embodiments 38-40, for treating a disease or condition associated with ocular angiogenesis.
Embodiment 49: The compound of any one of Embodiments 1-37, or a pharmaceutically acceptable salt thereof, or a stereoisomer thereof, or a hydrate or a solvate thereof, or the pharmaceutically composition of any one of Embodiments 38-40, for inhibiting choroidal angiogenesis.
Embodiment 50: The compound of any one of Embodiments 1-37, or a pharmaceutically acceptable salt thereof, or a stereoisomer thereof, or a hydrate or a solvate thereof, or the pharmaceutically composition of any one of Embodiments 38-40, for treating a disease or condition associated with choroidal angiogenesis.
Embodiment 51: The compound of any one of Embodiments 1-37, or a pharmaceutically acceptable salt thereof, or a stereoisomer thereof, or a hydrate or a solvate thereof, or the pharmaceutically composition of any one of Embodiments 38-40, for treating wet macular degeneration, diabetic retinopathy, or neovascular glaucoma.
Embodiment 52: The compound of any one of Embodiments 1-37, or a pharmaceutically acceptable salt thereof, or a stereoisomer thereof, or a hydrate or a solvate thereof, or the pharmaceutically composition of any one of Embodiments 38-40, for inhibiting a VEGFR2 kinase activity in a cell or a subject, wherein the inhibitory effect on the VEGFR2 kinase activity is at least or about 1.5 times, at least or about 2 times, at least or about 2.5 times, at least or about 3 times, or more of the inhibitory effect of compound

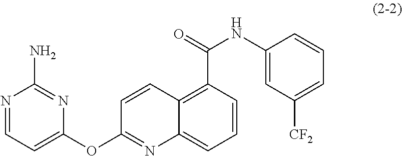

on the VEGFR2 kinase activity.
Embodiment 53: A method of treating a disease or condition associated with abnormal angiogenic cell proliferation or angiogenesis in a subject in need thereof, comprising: administering an effective amount of the compound of any one of Embodiments 1-37, or a pharmaceutically acceptable salt thereof, or a stereoisomer thereof, or a hydrate or a solvate thereof, or the pharmaceutically composition of any one of Embodiments 38-40, to the subject.
Embodiment 54: A method of treating a disease or condition associated with ocular angiogenesis in a subject in need thereof, comprising: administering an effective amount of the compound of any one of Embodiments 1-37, or a pharmaceutically acceptable salt thereof, or a stereoisomer thereof, or a hydrate or a solvate thereof, or the pharmaceutically composition of any one of Embodiments 38-40, to the subject.
Embodiment 55: A method of treating a disease or condition associated with choroidal angiogenesis in a subject in need thereof, comprising: administering an effective amount of the compound of any one of Embodiments 1-37, or a pharmaceutically acceptable salt thereof, or a stereoisomer thereof, or a hydrate or a solvate thereof, or the pharmaceutically composition of any one of Embodiments 38-40, to the subject.
Embodiment 56: A method of treating wet macular degeneration, diabetic retinopathy, or neovascular glaucoma in a subject in need thereof, comprising: administering an effective amount of the compound of any one of Embodiments 1-37, or a pharmaceutically acceptable salt thereof, or a stereoisomer thereof, or a hydrate or a solvate thereof, or the pharmaceutically composition of any one of Embodiments 38-40, to the subject.
Embodiment 57: The method of any one of Embodiments 53-56, wherein the subject has not been treated with compound

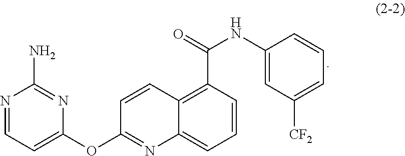

Embodiment 58: The method of any one of Embodiments 53-56, wherein the subject has been treated with compound

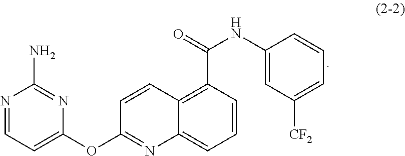

Embodiment 59: The method of any one of Embodiments 53-56, wherein the subject is more responsive to the compound of any one of Embodiments 1-37, or a pharmaceutically acceptable salt thereof, or a stereoisomer thereof, or a hydrate or a solvate thereof, or the pharmaceutically composition of any one of Embodiments 38-40, compared to compound

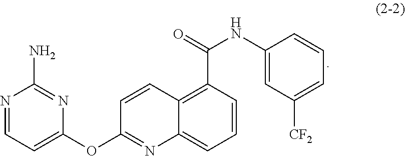

Embodiment 60: The method of Embodiment 59, wherein the subject is at least or about 1.5 times, at least or about 2 times, at least or about 2.5 times, at least or about 3 times, or more responsive to the compound of any one of Embodiments 1-15 compared to compound

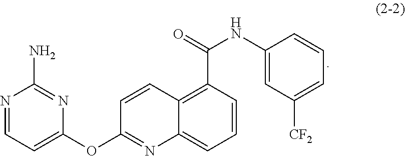

Embodiment 61: A method of improving the efficacy of compound

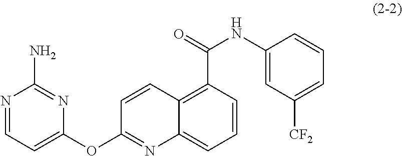

in inhibiting VEGFR2 protein kinase, comprising substituting at least one hydrogen in compound 2-2 with deuterium.
Embodiment 62: The method of Embodiment 61, wherein the hydrogen is a hydrogen connected to an aromatic ring or aromatic bicyclic ring in compound 2-2.
Embodiment 63: The method of Embodiment 62, wherein the hydrogen is at the para position of the —CF₃ group.
Embodiment 64: A method of treating cancer in a subject in need thereof, comprising: administering an effective amount of the compound of any one of Embodiments 1-37, or a pharmaceutically acceptable salt thereof, or a stereoisomer thereof, or a hydrate or a solvate thereof, or the pharmaceutically composition of any one of Embodiments 38-40, to the subject.
Embodiment 65: The method of Embodiment 64, wherein the cancer is lung cancer or liver cancer.
Embodiment 66: The method of Embodiment 64 or 65, wherein the cancer is NSCLC or HCC.
Embodiment 67: The method of any one of Embodiments 64-66, wherein the subject is not treated with a radiation therapy.
Embodiment 68: The method of any one of Embodiments 64-66, wherein the subject is treated with a radiation therapy.
Embodiment 69: The method of Embodiment 68, wherein the subject is treated with the radiation therapy before or concurrently with treatment with the compound of any one of Embodiments 1-37, or a pharmaceutically acceptable salt thereof, or a stereoisomer thereof, or a hydrate or a solvate thereof, or the pharmaceutically composition of any one of Embodiments 38-40.
Embodiment 70: The method of Embodiment 69, wherein treatment with the compound or the pharmaceutically composition ameliorates a side effect of the radiation therapy, and the side effect is optionally inflammation, edema, and/or cell death in a brain tissue of the subject.

### EXAMPLES

### Example 1. Synthesis of Compound 1

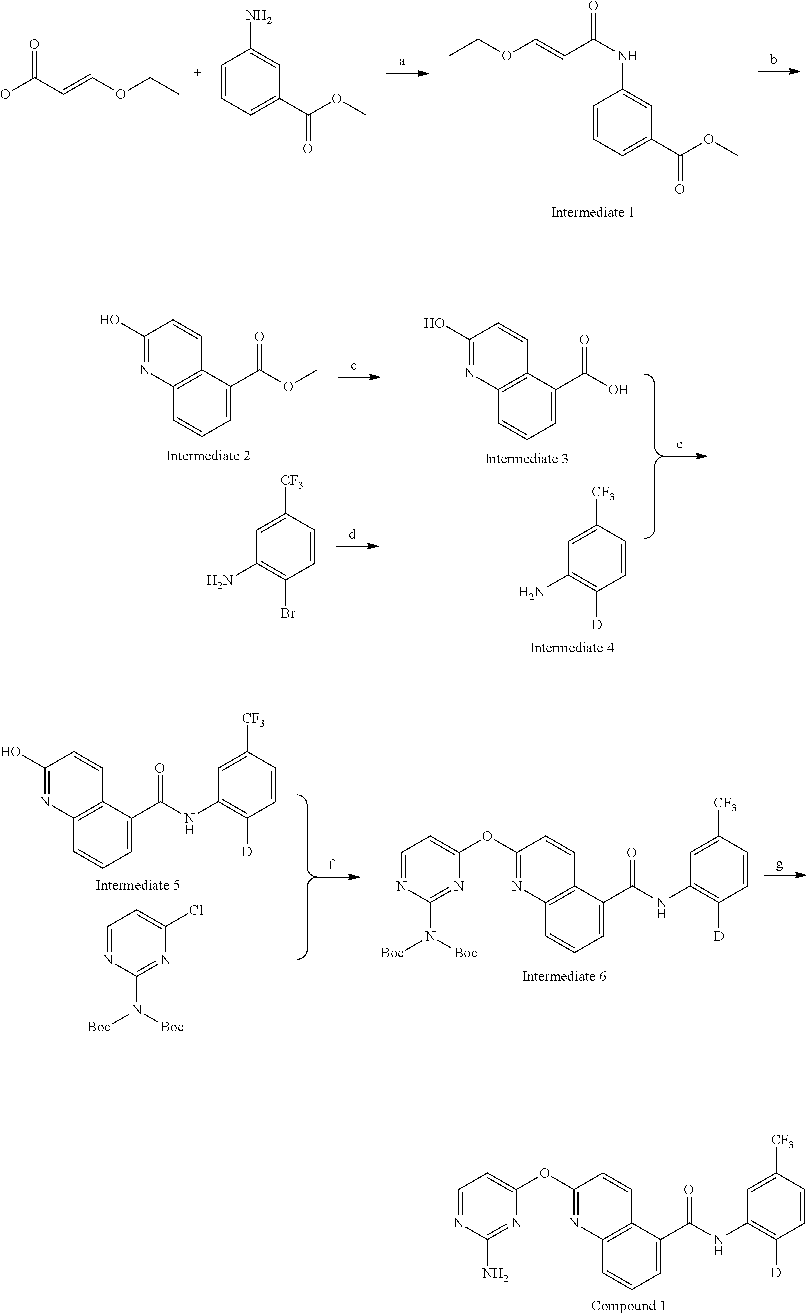

### Step 1. Preparation of Intermediate 1-Methyl 3-(3-ethoxyacrylamido)benzoate

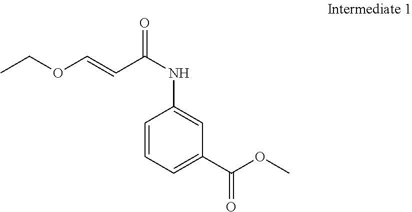 [SMILES: CCO/C=C/C(=O)Nc1cccc(C(=O)OC)c1]

To a solution of methyl 3-aminobenzoate (10 g, 66 mmol) dissolved in dichloromethane (DCM, 100 mL), pyridine (9.06 g, 119 mmol) was added. To the mixture, 3-ethoxyacryloyl chloride (15 g, 112 mmol) dissolved in DCM was then added at 0° C. The mixture was heated to room temperature and stirred for 2.5 hours. The reaction was monitored via TLC. After the reaction was completed, the mixture was washed with water and then with brine, dried with anhydrous Na₂SO₄ and concentrated to obtain a crude product. The product was purified by chromatography to obtain a purified Intermediate 1 (9.7 g, yield: 59%) as a white solid. MS (ESI) m/z: 272.1 [M+Na]⁺.

### Step 2. Preparation of Intermediate 2-2-Hydroxyquinoline-5-carboxylic acid methyl ester

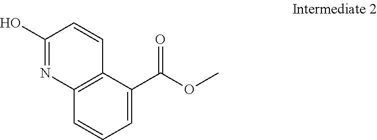

To a 175 mL concentrated H₂SO₄ Intermediate 1 (9.67 g, 0.039 mol) was added at 0° C. The reaction mixture was stirred at room temperature for 6 hours. The reaction was monitored via TLC. After the reaction was completed, the mixture was poured into ice-water. The precipitate was filtrated and washed with diethyl ether (Et₂O), recrystallized with ethanol, to obtain Intermediate 2 (2 g, yield: 25%) as a beige solid. MS (ESI) m/z: 204.1 [M+H]⁺.

### Step 3. Preparation of Intermediate 3-2-hydroxyquinoline-5-carboxylic acid

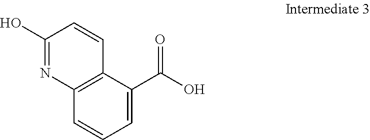 [SMILES: O=C(O)c1cccc2nc(O)ccc12]

To a solution of Intermediate 1 (2.0 g, 9.85 mmol) in methanol (MeOH, 20 mL) 2N NaOH (24.6 mL, 49.2 mmol) was added dropwise at 0° C. under stirring. Then the reaction mixture was stirred at room temperature overnight. The reaction was monitored via TLC. After the reaction was completed, the mixture was evaporated, and the residue was acidified by adding 1N HCl to pH 2. Precipitate was formed and collected by filtration, and dried to obtain a purified Intermediate 3 (0.8 g, 43%) as a white solid, which was directly used for the next step. MS (ESI) m/z: 190.1 [M+H]⁺.

### Step 4. Preparation of Intermediate 4-3-(trifluoromethyl) 6-deuterium-aniline

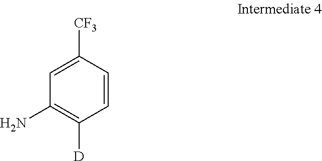 [SMILES: [2H]c1ccc(C(F)(F)F)cc1N]

2-bromo-5-trifluoromethylaniline (4.8 g, 20 mmol) was dissolved in tetrahydrofuran (40 mL) and the reaction system was purged with Ar. The mixture was stirred under −78° C. for 5 minutes and then n-butyl lithium (2.5 M, 32 mL, 80 mmol) was added to the mixture. After adding all n-butyl lithium, the reaction flask was warmed to room temperature and stirred at room temperature for 30 minutes. As the reaction system turned dark, it was moved back to −78° C. and stirred under −78° C. for another one hour. The reaction was monitored via TLC. After the reaction was completed, D₂O was added dropwise and the reaction was slowly quenched. Once the quenching was completed, the reaction mixture was warmed up to room temperature and stirred. The mixture was then filtered by diatomaceous earth and eluted with ethyl acetate. The filtrate was concentrated and purified using chromatography to obtain a purified Intermediate 4 as yellow oil (4 g, yield: 85%). ¹H NMR (400 MHz, Chloroform-d) δ 7.2 (d, J=7.7 Hz, 1H), 6.9 (d, J=7.9 Hz, 1H), 6.8 (s, 1H), 3.8 (s, 2H).

### Step 5. Preparation of Intermediate 5-2-hydroxy-N-(3-(trifluoromethyl)phenyl-6-deuterium)quinoline-5-carboxamide

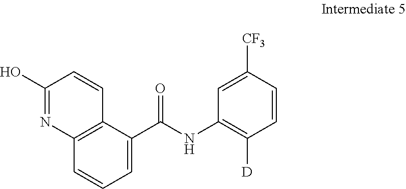 [SMILES: [2H]c1ccc(C(F)(F)F)cc1NC(=O)c1cccc2nc(O)ccc12]

To the reaction flask was added Intermediate 3 (1.89 g, 10 mmol) dissolved in N,N-dimethylformamide (100 mL) and triethylamine (2.02 g, 20 mmol). Under stirring, O-(7-azabenzotriazol-1-yl)-N,N,N′,N′-tetramethyluronium hexafluorophosphate (4.94 g, 13 mmol) was added, and then the reaction mixture was kept under 40° C. for 2 days. The reaction was monitored via TLC. After the reaction was completed, the reaction mixture was concentrated, and the residue was separated and purified by column chromatography to obtain Intermediate 5 (a white solid, 950 mg, with a yield of 29%). ¹H NMR (400 MHz, DMSO-d₆) δ 12.0 (s, 1H), 10.9 (s, 1H), 8.3 (s, 1H), 8.1 (d, J=9.8 Hz, 1H), 7.7-7.6 (m, 2H), 7.5-7.4 (m, 3H), 6.6 (dd, J=9.9, 1.9 Hz, 1H). MS (ESI) m/z: 334.2 [M+H]⁺.

### Step 6. Preparation of Intermediate 6-2-((2-[bis(tert-butoxycarbonyl)]aminopyrimidin-4-yl)oxy)-N-(3-(trifluoromethyl)phenyl-6-deuterium)quinoline-5-methanol amide

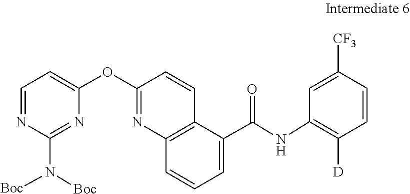 [SMILES: [2H]c1ccc(C(F)(F)F)cc1NC(=O)c1cccc2nc(Oc3ccnc(N(C(=O)OC(C)(C)C)C(=O)OC(C)(C)C)n3)ccc12]

To a reaction flask was added Intermediate 5 (666 mg, 2 mmol), 4-chloro-2-[bis(tert-butoxycarbonyl)amino]pyrimidine (725 mg, 2.2 mmol), cesium carbonate (980 mg, 3 mmol). The mixture was dissolved in dimethyl sulfoxide (15 mL), and reacted at 60° C. for 8 hours. The reaction was monitored via TLC. After the reaction was completed, the reaction mixture was cooled down to room temperature. 150 mL of water was added and the mixture was extracted with ethyl acetate. The organic phases were combined, dried over anhydrous sodium sulfate, filtered, and the filtrate was concentrated and purified by column chromatography to obtain Intermediate 6 (yellow Solid, 600 mg, 50% yield). ¹H NMR (400 MHz, DMSO-d₆) δ 11.0 (s, 1H), 8.9-8.8 (m, 2H), 8.3 (s, 1H), 8.1-7.9 (m, 2H), 7.9-7.9 (m, 1H), 7.6 (d, J=7.8 Hz, 1H), 7.6-7.5 (m, 2H), 7.4 (d, J=5.7 Hz, 1H), 1.3 (s, 18H). MS (ESI) m/z: 627.2 [M+H]⁺.

### Step 7. Preparation of Compound 1-2-((2-aminopyrimidin-4-yl)oxy)-N-(3-(trifluoromethyl)phenyl-6-deuterium)quinoline-5-carboxamide

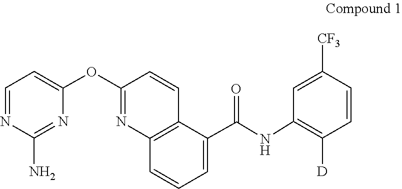 [SMILES: [2H]c1ccc(C(F)(F)F)cc1NC(=O)c1cccc2nc(Oc3ccnc(N)n3)ccc12]

To a reaction flask was added Intermediate 6 (600 mg, 1 mmol) dissolved in 6 mL dichloromethane. Then 6 mL of trifluoroacetic acid was added to the flask and the mixture was reacted at room temperature for 1 hour. The reaction was monitored via TLC. After the reaction was completed, ice water was added and the pH value was adjusted to 7-8 with saturated sodium bicarbonate. The organic layer was separated, washed with brine, dried over anhydrous sodium sulfate, and filtered. The filtrate was concentrated and purified by column chromatography to obtain Compound 1 (white solid, 250 mg, yield 62%). ¹H NMR (400 MHz, DMSO-d₆) δ 11.0 (s, 1H), 8.8 (d, J=9.1 Hz, 1H), 8.3 (s, 1H), 8.2 (d, J=5.5 Hz, 1H), 8.0 (d, J=8.3 Hz, 1H), 7.9 (d, J=6.9 Hz, 1H), 7.9-7.8 (m, 1H), 7.6 (d, J=7.8 Hz, 1H), 7.5 (d, J=7.9 Hz, 1H), 7.4 (d, J=9.1 Hz, 1H), 6.7 (s, 2H), 6.4 (d, J=5.5 Hz, 1H). MS (ESI) m/z: 427.2 [M+H]⁺.

### Example 2. VEGF Inhibition Experiment

### Cell Culture

The human HUVEC cell line (Sabic) were cultured in 10% fetal bovine serum HUVEC culture medium (Sabic) in six-well plates. After the cultured cells reached 90% confluency, they were subjected to serum starvation by using serum-free HUVEC culture medium for 6 hours. VEGF (R&D) was dissolved in HUVEC culture medium containing 10% fetal bovine serum to a concentration of 50 ng/mL. Drugs were dissolved in the VEGF solution to obtain drug mixture solutions of 1 nM, 10 nM, and 100 nM, respectively, and incubated at room temperature for 30 minutes. The serum-free HUVEC culture medium in the cell culture dish was aspirated after serum starvation, and the drug mixture solution incubated at room temperature was added to stimulate the cells for 5 minutes. The drug mixture solution was aspirated and the plate wells were washed with PBS for 3 times. Cell lysis solution was added to extract proteins.

### Western Blot Procedure

Electrophoresis: 1 L of electrophoresis buffer with ddH₂O was prepared. The bottom sealing film of the preset gel was teared off. The gel was assembled and pushed down to prevent leakage and tilting. The preset gel was rinsed with ddH₂O and checked for leakage. The inner groove was filled with electrophoresis buffer and removed the comb.

20 μg protein sample was added by inserting the pipette tip into the well to prevent sample drift. Markers to the two sides of the well were added. The inner groove was filled with buffer and the outer groove was filled to the corresponding mark. The system was run at a constant voltage of 80V until the sample forms a straight line, then the voltage was increased to 110V as needed.

### Transfer and Blocking

A transfer buffer was prepared by adding 100 mL of 10× transfer buffer and 200 mL of methanol and making the mixture up to 1 L with water. The gel cassette was opened and the gel was cut according to the marker. The gel could be briefly soaked in the transfer buffer.

The length and width of the gel (fixed width is 8.5 cm) were measured and a PVDF membrane of appropriate size was cut. The membrane was activated in methanol for at least 1 minute and then put in the transfer buffer for later use.

For assembling the transfer sandwich, the black pad was put at the bottom. The layer order was black pad, sponge, filter paper ×2, gel, membrane, filter paper ×2, sponge, and clear pad. Any bubbles between each layer were removed and the sandwich was rolled with a roller to eliminate air bubbles after placing the last sponge. The sandwich was put into the transfer tank, ice packs were added, and the transfer tank was filled with transfer buffer. The transfer tank was placed in a larger container with an ice slurry.

The proteins were transferred under the conditions of 300 mA for 70 minutes.

50 mL of blocking buffer (5% BSA) was prepared. The buffer was generally shook and block at room temperature for 2 hours.

Incubation with Primary and Secondary Antibodies

The membrane was wrapped with cling film on both sides and the membrane was cut with a surgical scalpel according to the target bands (230 kDa and 37 kDa). The membrane was incubated with the primary antibody overnight at 4° C. The primary antibody dilution buffer contain 5% BSA. Phospho-VEGF Receptor 2 (Tyr1175) (19A10) Rabbit mAb (CST2478S) is diluted 1000 times; and GAPDH (14C10) Rabbit mAb (CST2118S) is diluted 2000 times.

The primary antibody was removed and the membrane was rinsed with 5‰ TBST solution 3 times, 5 minutes each time. The membrane was incubated with the secondary antibody (Anti-rabbit IgG, HRP-linked Antibody, CST7074P2) at room temperature for 1 hour. The secondary antibody dilution buffer contained 5% BSA and was diluted 3000 times. The membrane was washed with 5‰ TBST solution 3 times, 5 minutes each time.

### Exposure

Equal volumes of ECL and AB reagents (Yeasen, 36208ES76) were mixed thoroughly, then evenly dripped onto the membrane. The membrane was then imaged with Chemiluminescence imaging system.

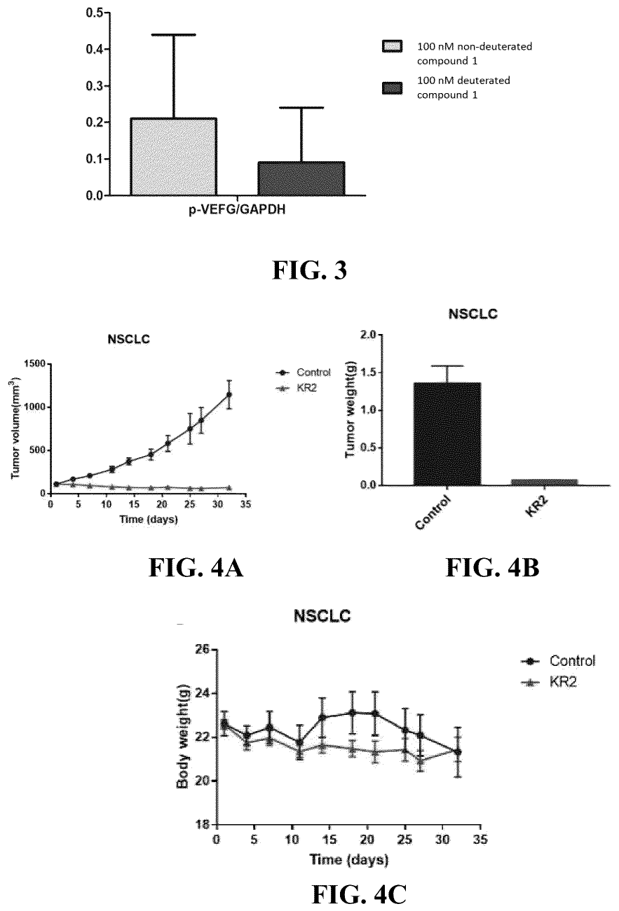

The results are shown in FIG. 2 and FIG. 3. As shown in FIG. 2, primary human HUVEC Cell line treated with human VEGF, or VEGF plus various concentration of Compound 1 at 1 nM, 10 nM, and 100 nM showed dose dependent inhibition of autophosphorylation of VEGF receptor2/KDR when treated with Compound 1. As shown in FIG. 3, deuterated Compound 1 exhibited higher VEGFR inhibition efficacy compared to undeuterated counterpart of Compound 1.

### Example 3. The Therapeutic Efficacy of Compound HY-3-2-H (Also Referred to as Compound KR2 or ANT-VI-4-1) in NSCLC and HCC PDX Models

### I. Methods

### Patients and Tissue Specimens

Fresh tumor specimens were obtained from patients with diagnosed non-small cell lung cancer (NSCLC) and hepatocellular carcinoma (HCC), respectively.

### Animals

Female BALB/c nude mice (4-5 week) were bought from Charles River Laboratory Animal Technology (Beijing, China). All the mice were housed in cages with 12 h light and fed ad libitum.

### Establishment of Patient-Derived Xenograft (PDX) Model

The fresh tumor tissue was cut into pieces (3-5 mm³) and implanted subcutaneously into the flank of immunodeficient mice by trocar within two hours. After tumor volume reached 1000 mm³, tumor tissue was removed and used for passage.

### II. Results

### Therapeutic Efficacy of Compound HY-3-2-H in NSCLC and HCC PDX Models.

The anti-tumor efficacy of Compound HY-3-2-H

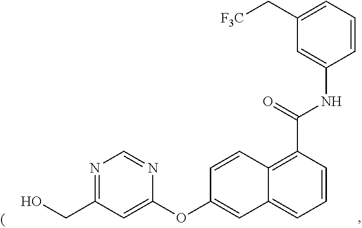 [SMILES: O=C(Nc1cccc(CC(F)(F)F)c1)c1cccc2cc(Oc3cc(CO)ncn3)ccc12]

also referred to as Compound KR2 or ANT-VI-4-1) was evaluated in NSCLC and HCC PDX models, respectively. As shown in FIG. 4A, Compound HY-3-2-H treatment significantly inhibited the tumor growth in NSCLC PDX model, compared to the normal saline control group (P<0.05). At the end of the experiment, tumor tissue was excised and weighed for tumor weight. The tumor weight was markedly reduced in HY-3-2-H treated group when compared to the control group (P<0.05, FIG. 4B). In addition, no obvious changes in general status and body weight were observed in the mice treated with Compound HY-3-2-H (FIG. 4C).

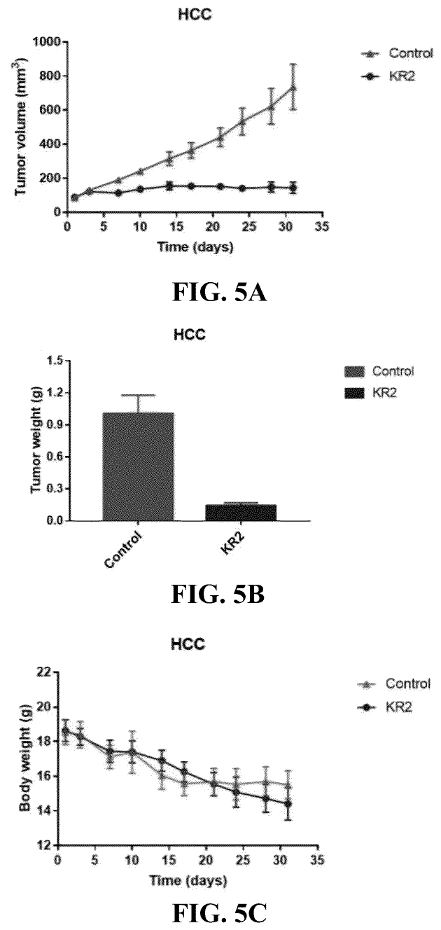

Similarly, Compound HY-3-2-H also exhibited potent anti-tumor effect in HCC PDX model. Compared to normal saline control, HY-3-2-H significantly inhibited the tumor growth (P<0.05, FIG. 5A) and reduced the tumor volume at the end of the experiment (P<0.05, FIG. 5B). There was no significant difference in body weight between HY-3-2-H treatment group and control group (FIG. 5C).

### Example 4. The Therapeutic Efficacy of Compound HY-3-2-H (Also Referred to as Compound KR 2 or ANT-VI-4-1) in Anti-VEGF Comparative Therapy on Radiation Induced Brain Tissue Damage

### I. Methods

### Irradiation Induced Brain Tissue Damage in BALB/c Mice

Prior to irradiation, healthy mice of the appropriate age are selected, and baseline measurements, including initial weight and general health status, were recorded.

Anesthesia was administered to minimize stress. Mice were positioned in an irradiator to ensure targeted brain area was uniformly exposed for a desired dose of radiation.

Immediately following irradiation, the mice were closely monitored for signs of distress or adverse effects. Supportive care, such as hydration gels, warmers, and easy access to food and water, was provided as needed. Long-term monitoring involves daily checks of weight, behavior, and general health, with antibiotics administered as needed to prevent infections.

Anti-VEGF Comparative Therapy with Bevacizumab or Compound ANT-VI-4-1 in Irradiated BALB/c Mice

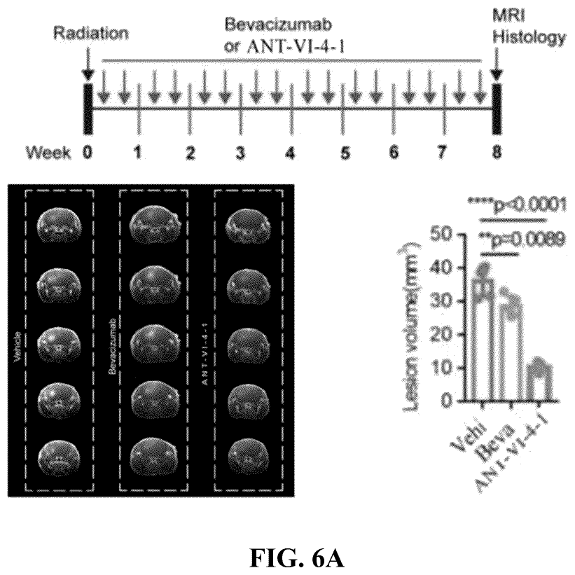

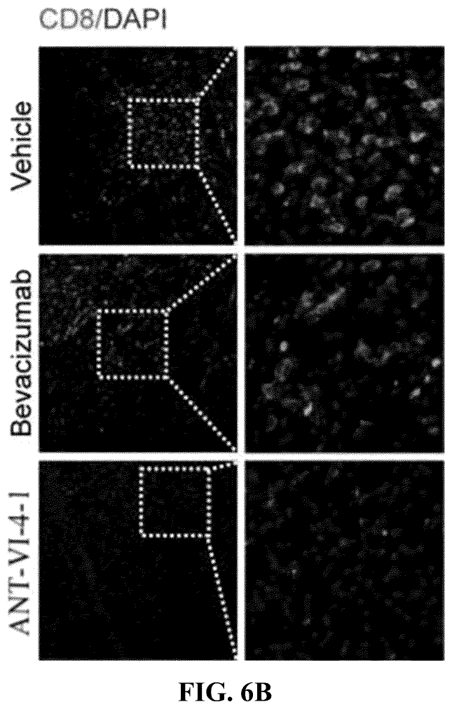

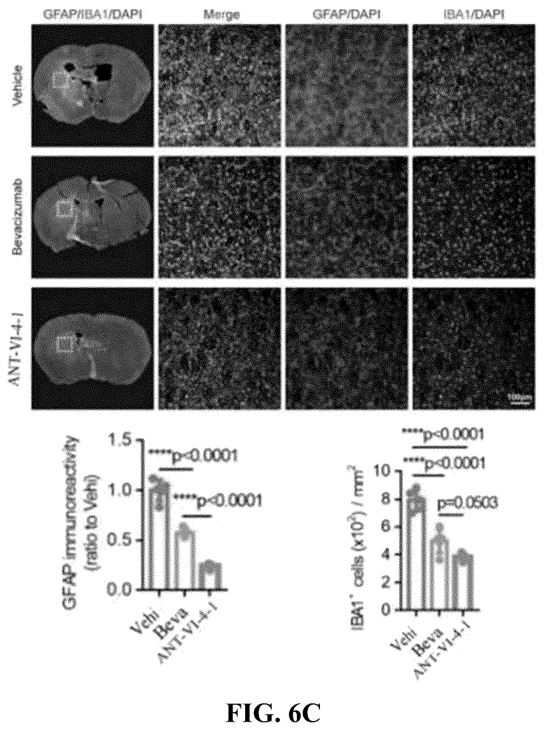

Following the irradiation protocol, the irradiated mice were separated into three groups. Each group was administered either saline (control group, labeled as Vehicle), bevacizumab (bevacizumab test group, labeled as Bevacizumab), or compound ANT-VI-4-1 (ANT-VI-4-1 test group, labeled as ANT-VI-4-1) via tail vein injection twice per week, as shown in FIG. 6A. After continuous treatment for 8 weeks, the mice were sacrificed. Appropriate tissues were then collected and analyzed in subsequent immunohistological studies.

### II. Results

### Therapeutic Efficacy of Compound HY-3-2-H in Anti-VEGF Comparative Therapy on Radiation Induced Brain Tissue Damage Models.

The therapeutic efficacy of compound HY-3-2-H (also referred to as Compound KR2 or ANT-VI-4-1) in anti-VEGF comparative therapy on radiation induced brain tissue damage was evaluated comprehensively in animal models.

As shown in the anti-VEGF comparative therapy in FIG. 6B, the lesion volume, as indicated by the presence of infiltrating CD8⁺ T cells, was greatly reduced when the mice were treated with compound ANT-VI-4-1. Nearly all lesions in the irradiation-damaged brain tissues were repaired after treatment with compound ANT-VI-4-1 via tail vein injection twice per week after 8 weeks. In comparison, anti-VEGF therapy with bevacizumab only partially (approximately 50% as effective as the treatment with compound ANT-VI-4-1) repaired the irradiation-damaged brain tissues, while the control group remained extensively damaged.

The therapeutic efficacy of compound ANT-VI-4-1 were also measured by GFAP (Glial Fibrillary Acidic Protein) and IBA1 (Ionized Calcium-Binding Adapter Molecule 1) immunoreactivities, as shown in FIG. 6C. The presence of infiltrating microglia and macrophages were both greatly reduced when the mice were treated with compound ANT-VI-4-1. Taken together with FIG. 6B and FIG. 6C, the compound ANT-VI-4-1 was highly effective at repairing irradiation-damaged brain tissues.

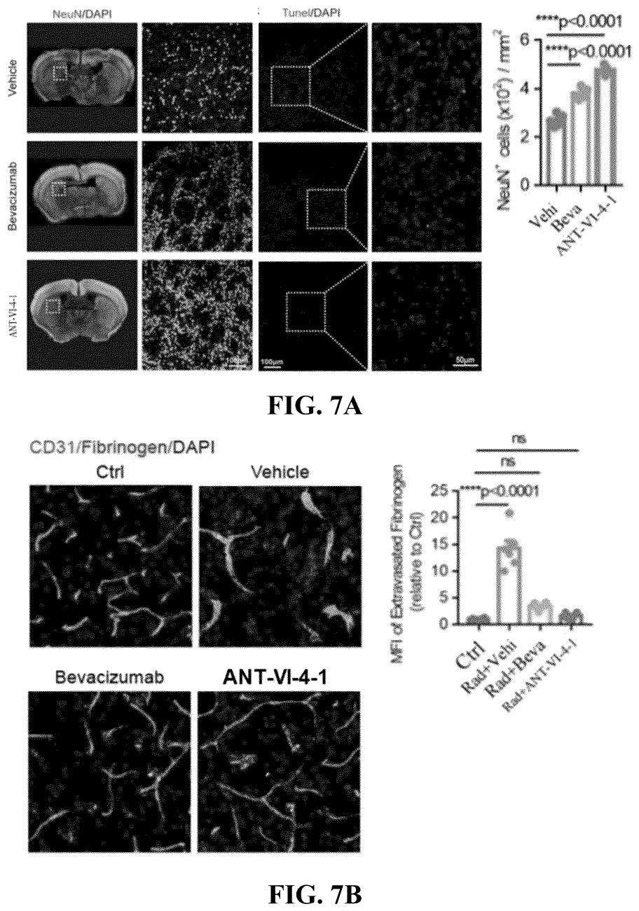

NeuN (Neuronal Nuclei) immunoreactivity for surviving neurons and TUNEL (Terminal deoxynucleotidyl transferase dUTP Nick End Labeling) immunoreactivity for DNA fragmentation were also measured in the anti-VEGF comparative therapy in FIG. 7A in order to compare the therapeutic efficacies of the compound ANT-VI-4-1 or anti-VEGF antibody Bevacizumab after radiation. The highest presence of surviving neurons and the least amount of DNA fragmentation indicated that the compound ANT-VI-4-1 was highly effective at repairing irradiation-damaged brain tissues, better than the anti-VEGF therapy with Bevacizumab.

FIG. 7B showed the therapeutic efficacy of the compound ANT-VI-4-1, when measured by the mean fluorescence intensity (MFI) of extravasated fibrinogen immunoreactivity for hemostasis activity after radiation. In this experiment, the compound ANT-VI-4-1 not only showed the lowest MFI of extravasated fibrinogen immunoreactivity but also restored hemostasis activity to levels comparable to normal, undamaged brain tissues.

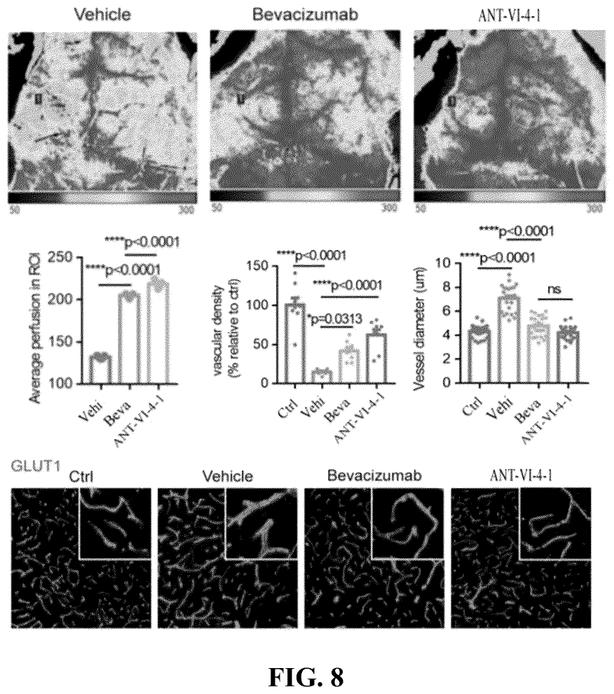

FIG. 8 showed the therapeutic efficacy of the compound ANT-VI-4-1, when measured by GLUT1 (Glucose Transporter Type 1) immunoreactivity for blood flow recovery and proper vascular network formation after radiation. Again, the compound ANT-VI-4-1 was not only effective at promoting blood flow recovery and proper vascular network formation, but also restore vascular network to levels comparable to normal, undamaged brain tissues.

In conclusion, the compound ANT-VI-4-1 was highly effective at repairing irradiation-damaged brain tissues, better than the anti-VEGF therapy with Bevacizumab. The results show that ANT-VI-4-1 led to reduction in inflammation, reduction in edema, and reduction in cell death in brain tissues following irradiation. In one aspect, ANT-VI-4-1 attenuated necrotic lesion size and gliosis induced by radiation (see, e.g., FIGS. 6A-6C). In one aspect, ANT-VI-4-1 alleviated neuronal loss and fibrinogen exudation after radiation (see, e.g., FIGS. 7A-7B). In another aspect, ANT-VI-4-1 ameliorated blood flow recovery and proper vascular network formation after radiation (see, e.g., FIG. 8).

### Example 5. The Inhibitory Effect of Compounds on VEGF-Stimulated Phosphorylation of VEGFR2/KDR Protein

This example shows phosphorylation of VEGFR2/KDR protein was significantly inhibited by Compounds KDR2-2 (also referred to as Compound 2-2), HY-3-2-H, and Compound 1.

Materials: 6-well plate, HUVEC cell, PBS, drug solution, recombinant Human VEGF 165 Protein, β-Actin (13E5) Rabbit mAb, Phospho-VEGF Receptor 2 (Tyr1175) (19A10) Rabbit mAb, Anti-rabbit IgG, RIPA, Marker, 5× loading buffer, Protein phosphatase inhibitor mixture, MOPS running buffer, 10×trans buffer, non-fat powdered milk, TBST, 8% prefabricated adhesive, ECL enhanced chemiluminescence, ethanol, methanol, 45 μm PVEF membrane.

HUVEC cells were seeded into 6-well plates at a density of 2×10⁵ cells per well and cultured. Compound treatment was perform when the cells reached 70-80% confluence. The blank control group and the model group were treated with 1 mL of 0.5% DMSO solution, while the administration groups received equal volumes of test sample solutions (1000, 100, 10, 1 nM) of KDR2-2 (also referred to as Compound 2-2), Compound 1, or HY-3-2-H at varying concentrations, followed by 30 minutes of incubation. The model group and administration groups were treated with 300 nM VEGF (11.52 g/ml) for 10 minutes.

For protein extraction, supernatant was removed from all wells. Cells were washed twice with pre-chilled PBS. 150 μL of pre-chilled protein lysis buffer was added to each well, then the culture plate was placed on ice for lysis for 30 minutes. The lysate was transferred to centrifuge tubes to centrifuge at 4° C., 12,000 rpm for 15 minutes. The supernatant was collected for subsequent protein concentration measurement.

For Western blotting, the following procedures were used:
a. Preparation of Loading Samples: Based on the concentration measured by the BCA kit, dilute the samples to a uniform concentration. Add 5× loading buffer, mix well, and heat in a water bath for 5 minutes. Load 15 μl per sample (concentration: 15-20 μg).
b. Using YaMei 8% precast gels, load 15 μL of test samples per well alongside 5 μL of Marker per well. Initiate electrophoresis at an initial voltage of 80 V. After approximately 30 minutes, increase the voltage to 120 V and continue electrophoresis for an additional 60 minutes.
c. Perform protein transfer using the wet transfer method under the following conditions: 400 mA for 25 minutes with the apparatus placed on ice. After transfer is complete, cut the membrane according to the molecular weight marker to isolate the target protein bands. Incubate the membrane in TBST solution and wash with agitation three times, 10 minutes per wash.
d. Blocking: Place the washed PVDF membrane in TBST solution containing 5% milk, and block at room temperature for 2 hours.
e. Primary Antibody Incubation: Incubate the membrane with primary antibody on a shaker at 4° C. overnight. Subsequently, wash the membrane four times with TBST, 15 minutes per wash.
f. Secondary Antibody Incubation: Incubate with secondary antibody at room temperature for 1 hour, followed by four 15-minute washes with TBST.
g. Detection: Under light-protected conditions, mix ECL Plus ultrasensitive chemiluminescent substrate solutions A and B in equal volumes. Capture images of target protein bands using a gel imaging system. Analyze protein band images with ImageJ software to quantify mean gray values. Statistics: One-way ANOVA (Prism 8.0.2); data presented as mean #SD. Significance: P<0.05 (denoted as “*”).

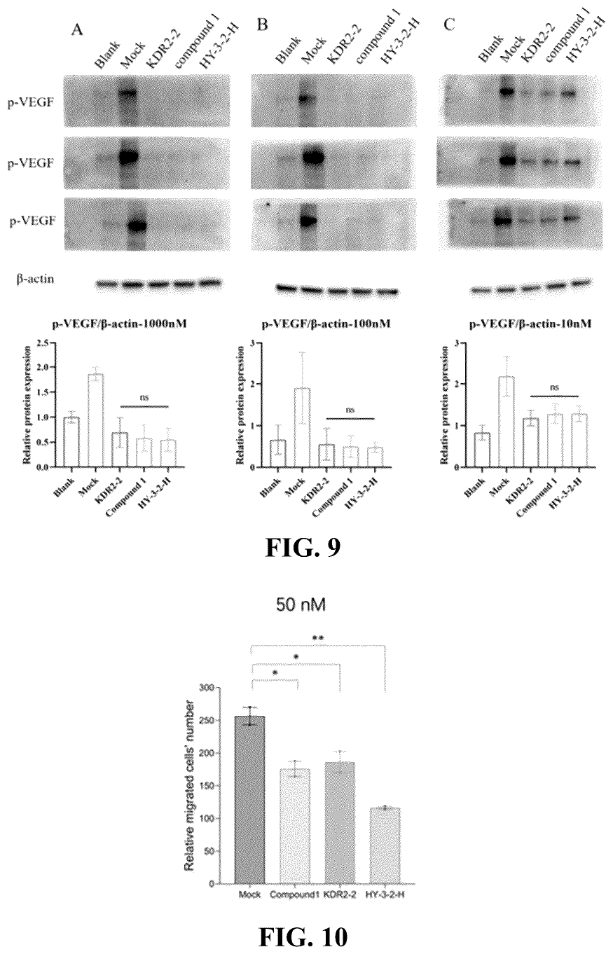

The results are shown in FIG. 9, showing compared to the Blank group, VEGF stimulation significantly increased phosphorylation of VEGFR in HUVECs in the Mock group. Phosphorylation of VEGFR2/KDR was significantly inhibited by Compound 2-2 (KDR2-2), HY-3-2-H, and Compound 1 from 10 to 1000 nM.

### Example 6. The Effect of Compounds on Cell Migration

This example investigated the effect of compounds on the migratory ability of Human Umbilical Vein Endothelial Cells (HUVEC) using the Transwell assay.

Materials: Transwell inserts, 24-well plate, fine tweezers, pipette tips, pipettes, 15 mL centrifuge tubes, Cell counting chamber, paper towels, ethanol, transfer pipettes, EP tubes, Serum-free cultivation medium, complete cultivation medium, PBS, HBSS, Drug, 0.125% trypsin, 4% PFA, 0.1% crystal violet, Microscope slides, distilled water, cotton swabs.

Methods: The following steps were performed.
(1) Pre-warm serum-free cultivation medium, complete cultivation medium, PBS, HBSS, and 0.125% trypsin in a 37° C. water bath. Retrieve cells from a 37° C. incubator with 5% CO₂, remove the complete cultivation medium, and wash once with HBSS. Add 0.125% trypsin and return to the incubator for 1 min. Observe under a microscope: if cells appear rounded and bright, add complete cultivation medium to stop digestion. Gently pipette the cell suspension to detach all adherent cells, transfer to a 15 mL centrifuge tube, and centrifuge at 1000 rpm for 5 min.
(2) Clean the cell counting chamber and cover slip with 75% ethanol, then wipe dry with paper towels. Examine under a microscope to ensure cleanliness for accurate counting.
(3) After centrifugation, aspirate the supernatant and resuspend the cell pellet in serum-free cultivation medium. Mix well by pipetting, then load 10 μL of the suspension into the counting chamber by introducing it along the edge of the cover slip until the counting area is filled.
(4) Calculate cell concentration based on the count and adjust to a density of 1×10⁵ cells/mL using serum-free cultivation medium.
(5) Add the drug at a final concentration of 50 nM and DMSO at 0.5% to the cell suspension, then mix. Place Transwell inserts into the 24-well plate, add 600 μL of complete cultivation medium to the lower chamber, and 200 μL of cell suspension to the upper chamber. Incubate the plate in a 37° C., 5% CO₂ incubator for 24 h.
(6) After 24 h, remove the inserts, discard the medium, and add 100 μL (upper chamber) and 600 μL (lower chamber) of 4% PFA for 30 min at room temperature for fixation.
(7) Remove the fixative and stain with 100 μL (upper chamber) and 600 μL (lower chamber) of 0.1% crystal violet for 20 min at room temperature.
(8) Rinse the stained inserts with distilled water, and gently wipe off non-migrated cells from the upper side of the membrane using a wet cotton swab.
(9) Capture images using an inverted phase-contrast fluorescence microscope at 50× and 100× magnification.

Migrated cells were counted using ImageJ software. One-way ANOVA was performed using Prism 8.0.2. Data are presented as mean±standard deviation (mean±SD). P-value<0.05 is considered statistically significant, denoted by “*”. FIG. 10 shows that compared to the mock group, treatment with HY-3-2-H, KDR2-2 (Compound 2-2), and Compound 1 reduced the number of migrating HUVEC cells, indicating that HY-3-2-H, KDR2-2, and Compound 1 can inhibit the migration of HUVEC cells (*P<0.05).

### Example 7. The Effect of Compounds on Vascular Tube Formation

This example investigated the effect of compounds on the in vitro vascular tube formation ability of HUVECs using a tube formation assay.

Materials: Growth factor-reduced Matrigel, 96-well plate, pipette tips, ice box, 15 mL centrifuge tubes, cell counting chamber, paper towels, ethanol, Transfer pipettes, EP tubes, serum-free medium, complete medium, PBS, HBSS, drug solutions, 0.125% trypsin.

Methods: The following steps were performed.
(1) Preparation of Matrigel-coated plates: Thaw aliquoted Matrigel overnight at 4° C. Pre-cool the 96-well plate and pipette tips (stored in 15 mL tubes) at −20° C.
(2) Cell starvation (optional): Pre-warm HBSS and serum-free medium at 37° C. Remove old medium from cell culture plates, wash with HBSS, and starve cells in serum-free medium for 3-6 h (skip if cell condition is suboptimal).
(3) Matrigel coating: Add 50 μL of chilled Matrigel per well using pre-cooled tips, ensuring even distribution without bubbles. Incubate at 4° C. for 30 min, then transfer to a 37° C., 5% CO₂ incubator for 30 min to solidify.
(4) Cell harvesting: Digest cells with 0.125% trypsin (1 min at 37° C., 5% CO₂), terminate with complete medium upon rounding (microscopic confirmation). Centrifuge cell suspension at 1000 rpm for 5 min.
(5) Cell counting: Clean counting chamber and cover slip with 75% ethanol, then load 10 μL cell suspension for counting.
(6) Cell suspension preparation: Adjust cell density to 3×10⁵ cells/mL in serum-free medium.
(7) Drug treatment: Add drugs (50 nM final concentration) or DMSO (0.5% final) to cell suspensions. Seed 100 μL/well onto Matrigel (triplicate wells per group).
(8) Incubation and imaging: Culture at 37° C., 5% CO₂ for 4 h. Capture images using an inverted phase-contrast microscope (5× magnification).

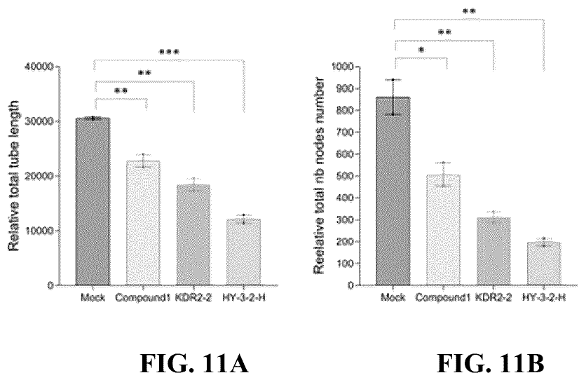

Tube nodes and total length quantified via ImageJ. Statistics: One-way ANOVA (Prism 8.0.2); data presented as mean±SD. Significance: P<0.05 (denoted as “*”). FIGS. 11A-11B show that treatment with HY-3-2-H, KDR2-2 (Compound 2-2), and Compound 1 significantly reduced both the total tube length (FIG. 11A) and number of nodes (FIG. 11B) in HUVEC cells compared to the mock group, indicating potent inhibition of tube formation capability (*P<0.05, **P<0.01).

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating a radiation induced brain tissue damage in a subject in need thereof, comprising administering to the subject a therapeutically effective amount of a compound, a pharmaceutically acceptable salt thereof, a deuterated compound thereof, a stereoisomer thereof, a hydrate thereof, or a solvate thereof, wherein the subject suffers from the radiation induced brain tissue damage and the compound is

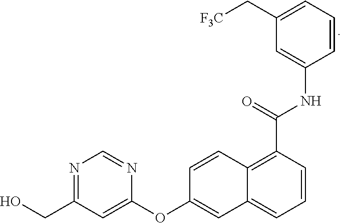 [SMILES: O=C(Nc1cccc(CC(F)(F)F)c1)c1cccc2cc(Oc3cc(CO)ncn3)ccc12]

2. The method of claim 1, wherein the administering reduces infiltrating CD8⁺ T cells in a lesion of the radiation induced brain tissue damage in the subject.

3. The method of claim 1, wherein the administering reduces infiltrating microglia in a lesion of the radiation induced brain tissue damage in the subject.

4. The method of claim 1, wherein the administering reduces infiltrating macrophages in a lesion of the radiation induced brain tissue damage in the subject.

5. The method of claim 1, wherein the administering promotes neuronal survival in a lesion of the radiation induced brain tissue damage in the subject.

6. The method of claim 1, wherein the administering reduces DNA fragmentation in a lesion of the radiation induced brain tissue damage in the subject.

7. The method of claim 1, wherein the administering reduces extravasated fibrinogen in a lesion of the radiation induced brain tissue damage in the subject.

8. The method of claim 1, wherein the administering promotes blood flow recovery in a lesion of the radiation induced brain tissue damage in the subject.

9. The method of claim 1, wherein the administering restores vascular network in a lesion of the radiation induced brain tissue damage in the subject.

10. The method of claim 1, wherein the administering reduces inflammation in a lesion of the radiation induced brain tissue damage in the subject.

11. The method of claim 1, wherein the administering reduces edema caused by the radiation induced brain tissue damage in the subject.

12. The method of claim 1, wherein the administering attenuates a necrotic lesion size and gliosis caused by the radiation induced brain tissue damage in the subject.

13. The method of claim 1, wherein the administering alleviates neuronal loss and fibrinogen exudation caused by the radiation induced brain tissue damage in the subject.

14. The method of claim 1, wherein the administering ameliorates blood flow recovery and vascular network formation in a lesion of the radiation induced brain tissue damage in the subject.

15. The method of claim 1, wherein the administering inhibits phosphorylation of VEGFR2/KDR in endothelial cells in a lesion of the radiation induced brain tissue damage in the subject.

16. The method of claim 1, wherein the administering inhibits endothelial cell migration in a lesion of the radiation induced brain tissue damage in the subject.

17. The method of claim 1, wherein the administering inhibits vascular tube formation of endothelial cells in a lesion of the radiation induced brain tissue damage in the subject.

18. The method of claim 1, wherein the pharmaceutically acceptable salt is selected from the group consisting of phosphate salt, D-camphorsulfonate salt, chloride salt, bromide salt, fluoride salt, sulfate salt, nitrate salt, formate salt, acetate salt, propionate salt, oxalate salt, malonate salt, succinate salt, fumarate salt, maleate salt, lactate salt, malate salt, tartrate salt, citrate salt, picrate salt, methanesulfonate salt, benzene mesylate salt, benzenesulfonate salt, aspartate salt, and glutamate salt.

19. The method of claim 1, wherein the radiation induced brain tissue damage is a side effect of a radiation therapy.

20. The method of claim 1, wherein the subject is treated with the radiation therapy before or concurrently with the administering of the compound, the pharmaceutically acceptable salt thereof, the stereoisomer thereof, the hydrate thereof, or the solvate thereof.
